(19) United States
(12) Patent Application Publication
(10) Pub. No.: US 2023/0397814 A1
(43) Pub. Date: Dec. 14, 2023

(54) DIGITAL TELEPATHY ECOSYSTEM METHOD AND DEVICES

(71) Applicants: Mahdi Orooji, Santa Clara, CA (US); Gaurav Kolhe, Milpitas, CA (US); Morteza Rafatirad, Tehran (IR)

(72) Inventors: Mahdi Orooji, Santa Clara, CA (US); Gaurav Kolhe, Milpitas, CA (US); Morteza Rafatirad, Tehran (IR)

(21) Appl. No.: 17/837,230

(22) Filed: Jun. 10, 2022

(51) Int. Cl. A61B 5/00; A61B 5/16; G06Q 20/38

(52) U.S. Cl. CPC A61B 5/0022; A61B 5/7275; A61B 5/0006; A61B 5/0008; A61B 5/165; A61B 5/7267; G06Q 20/389

(57) ABSTRACT

The embodiments disclose an apparatus including at least one wearable with a plurality of sensors configured to measure physiological signals of the user, an app on a user smart phone wirelessly coupled to the wearable configured for receiving and forwarding wearable measured physiological signals, a digital network cloud platform wirelessly coupled to the app configured for receiving and processing the measured physiological signals, at least one machine learning device coupled to the digital network cloud platform configured for analyzing the measured physiological signals, at least one artificial intelligence device coupled to the at least one machine learning device configured for determining indications of user well-being conditions, at least one computer coupled to the at least one machine learning and artificial intelligence device configured to transmit well-being conditions indications to the digital telepathy app on the user smart phone, and transmit the well-being conditions indications to persons selected by the user.

### BACKGROUND

Wearable electronics make it possible to monitor human activity and behavior. In the future interconnected world where the computing systems are ubiquitous and interleaved in our daily life, evidence-based, scientific automatic capturing and prediction of human emotion and well-being using computing technology will be crucial to allow wearable technology to interact with us in an uninterrupted, non-disturbing, and non-disruptive manner. Empowering the wearable technologies with advanced ML and AI to automatically capture and predict one's well-being such as stress, anxiety, sleep disorder, depression, lack of gratitude, and loneliness will be the enabler of the next disruptive technology in human-computer interaction allowing many new applications in areas such as health, well-being, social networking, gaming, and entertainment, artificial reality and virtual reality, just to name a few. Stress, an important indicator of well-being, is a major concern in the general population. At the same time, mobile application usage is continuously increasing such that on average individuals are spending several hours every day on their mobile browsing the web or using a particular set of apps. While some applications are essential for day-to-day activities, such as banking, many other applications are mostly perceived for entertainment and or pleasure. However, the real impact of these applications and their long usage/screen-time on stress while studied well in literature by the scientific community has not been brought into the commercial domain to change people's habits of using the technology. For instance, long usage of social media applications is shown to have a significant impact on individual stress and anxiety.

### BRIEF DESCRIPTION OF THE DRAWINGS

FIG. 1 shows for illustrative purposes only an example of an overview of a digital telepathy ecosystem of one embodiment.

FIG. 2 shows a block diagram of an overview flow chart of a digital telepathy ecosystem of one embodiment.

FIG. 3 shows a block diagram of an overview of a digital telepathy ecosystem of one embodiment.

FIG. 4 shows for illustrative purposes only an example of a digital telepathy ecosystem technology network cloud platform of one embodiment.

FIG. 5A shows a block diagram of an overview of a wearable of one embodiment.

FIG. 5B shows a block diagram of an overview of machine learning and artificial intelligence of one embodiment.

FIG. 5C shows a block diagram of an overview of intervention applications of one embodiment.

FIG. 6 shows for illustrative purposes only an example of a machine learning process of one embodiment.

FIG. 7 shows a block diagram of an overview of a detected/predicted emotion of one embodiment.

FIG. 8 shows a block diagram of an overview of social media interactions of one embodiment.

FIG. 9 shows a block diagram of an overview of empathy in the metaverse of one embodiment.

FIG. 10 shows for illustrative purposes only an example of a wearable band on the back of a cellphone of one embodiment.

FIG. 11 shows a block diagram of an overview of wearable bands for use by two persons of one embodiment.

FIG. 12 shows a block diagram of an overview of feeling the emotional state in real-time of one embodiment.

FIG. 13 shows for illustrative purposes only an example of cognitive empathy of one embodiment.

FIG. 14 shows a block diagram of an overview of the interactive sexual experiences of one embodiment.

FIG. 15 shows for illustrative purposes only an example of cryptocurrency rewards to digital telepathy active participants of one embodiment.

FIG. 16 shows for illustrative purposes only an example of advertising products and services related to targeted emotional states of one embodiment.

FIG. 17 shows a block diagram of an overview of selected persons providing a level of psychotherapy of one embodiment.

FIG. 18 shows a block diagram of an overview of monitoring adolescence stress and anxiety of one embodiment.

FIG. 19 shows a block diagram of an overview of evidence-based well-being and emotional data of teenagers of one embodiment.

FIG. 20 shows a block diagram of an overview of a primary care physician monitoring the physiological signals of a user of one embodiment.

FIG. 21 shows a block diagram of an overview of the user's demographic information of one embodiment.

FIG. 22 shows a block diagram of an overview of empowering the next generation of social networking/dating apps of one embodiment.

FIG. 23 shows a block diagram of an overview of emotional states integrated with an emoji of one embodiment.

FIG. 24 shows a block diagram of an overview of sharing well-being conditions indications with persons selected by the user of one embodiment.

FIG. 25 shows a block diagram of an overview of application integrated intervention of one embodiment.

FIG. 26A shows a block diagram of an overview of artificial intelligence of one embodiment.

FIG. 26B shows a block diagram of an overview of AI in two phases of one embodiment.

FIG. 26C shows a block diagram of an overview of AI in six steps of one embodiment.

FIG. 27A shows a block diagram of an overview of pre-processing of one embodiment.

FIG. 27B shows a block diagram of an overview of feature extraction of one embodiment.

FIG. 27C shows a block diagram of an overview of statistics features of one embodiment.

FIG. 27D shows a block diagram of an overview of signal extracted features of one embodiment.

FIG. 27E shows a block diagram of an overview of deep learning extracted features of one embodiment.

FIG. 28A shows a block diagram of an overview of the feature selection of one embodiment.

FIG. 28B shows a block diagram of an overview of the classification of one embodiment.

FIG. 28C shows a block diagram of an overview of an assessment of one embodiment.

FIG. 28D shows a block diagram of an overview of system adjustment of one embodiment.

FIG. 29 shows a block diagram of an overview of collecting user emotions of one embodiment.

FIG. 30 shows a block diagram of an overview of blockchain technology of one embodiment.

FIG. 31 shows a block diagram of an overview of NFTs of one embodiment.

FIG. 32 shows a block diagram of an overview of framework for emotional empathy of one embodiment.

FIG. 33 shows a block diagram of an overview of machine learning used to derive the emotional state of one embodiment.

FIG. 34 shows a block diagram of an overview of ecosystem chat rooms of one embodiment.

FIG. 35A shows a block diagram of an overview of output that may differ from one embodiment.

FIG. 35B shows a block diagram of an overview of empathy and emotion sharing of one embodiment.

FIG. 36A shows a block diagram of an overview of children's screen time using gaming consoles of one embodiment.

FIG. 36B shows a block diagram of an overview of teenverse artificial intelligence enabled emotional understanding of one embodiment.

### DETAILED DESCRIPTION OF THE INVENTION

In the following description, reference is made to the accompanying drawings, which form a part hereof, and which are shown by way of illustration as a specific example in which the invention may be practiced. It is to be understood that other embodiments may be utilized, and structural changes may be made without departing from the scope of the present invention.

### GENERAL OVERVIEW

It should be noted that the descriptions that follow, for example, in terms of a digital telepathy ecosystem method and devices are described for illustrative purposes and the underlying system can apply to any number and multiple types of emotional states. In one embodiment of the present invention, the digital telepathy ecosystem method and devices can be configured using a wearable with a plurality of sensors. The digital telepathy ecosystem method and devices can be configured to include wristbands and can be configured to include a band on the back of a cell phone using the present invention.

The digital telepathy ecosystem method and devices collect physiological data of users and user behavior in the user's normal life, as well as during the screen time of each application, and identify the level of well-being such as mindfulness, emotion, stress, fear, happiness, sadness, and anxiety during the normal life as well as during the screen time.

At least one user is a part of the ecosystem. In this case, the ecosystem monitors and provides intelligent feedback to the user. However, the ecosystem can be expanded to the people of two or more in the case of digital telepathy and to a broader group when dealing with a setting where hospitals/businesses are utilizing the data to offer services. For ads tailoring, it's one too many applications.

The framework comes with an SDK (Software Development Kit) for its adaptation into the metaverse. With the SDK, one can readily access the data from the wearable/phone devices using state-of-the-art APIs to deploy empathy in the evolving metaverse instantly. The SDK is compatible across all the platforms and can be deployed on-device or in-cloud based on the application type. The SDK for data collection and on-device learning can be used for federated learning styles where more than one model exists per given user and the aggregation algorithm can be carried out on the server. Based on the activity the user is performing, models can be deployed in reinforcement style to increase the accuracy of the prediction. Having one model for all the scenarios will not maximize the performance. For example, when driving, reinforcement learning can detect the user state as driving and deploy the model that has collected and leveraged more context information for better performance. The selection of models based on states and for maximizing rewards uses reinforcement style learning, while each model can be trained across user data using federated learning styles.

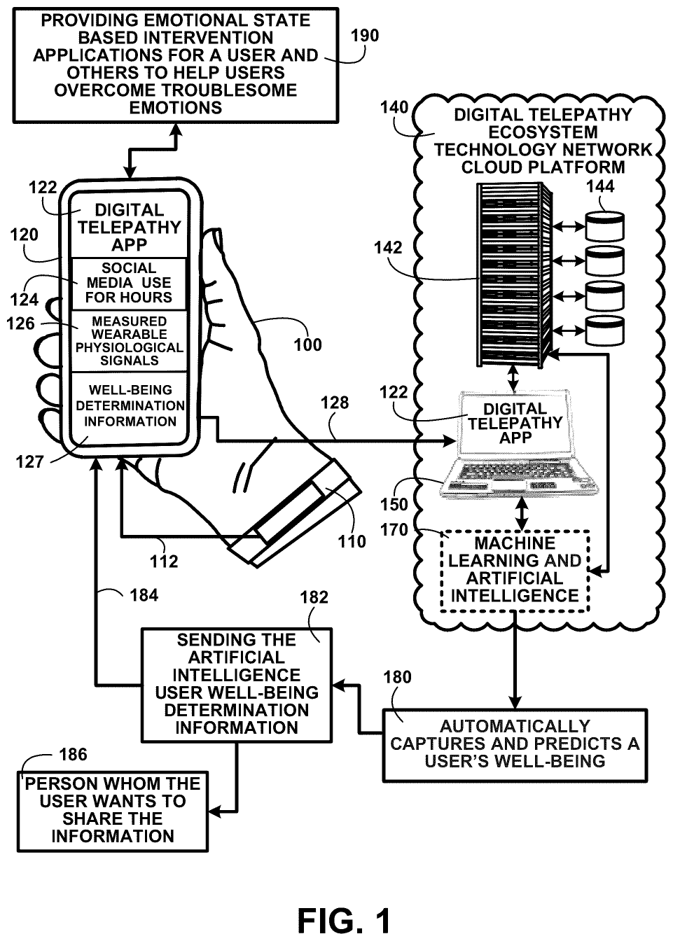

FIG. 1 shows for illustrative purposes only an example of an overview of a digital telepathy ecosystem of one embodiment. FIG. 1 shows a user 100 wearing a wearable 110 with machine learning and artificial intelligence 170 that is wirelessly coupled 112 to a user smart phone 120. The user smart phone 120 has installed a digital telepathy app 122 for sending social media use for hours 124, measured wearable physiological signals 126, and machine learning and artificial intelligence 170. The digital telepathy app 122 is used for the measured wearable physiological signals transmission 128 to a digital telepathy ecosystem technology network cloud platform 140. The digital telepathy ecosystem technology network cloud platform 140 includes at least one server 142, at least one database 144, a computer 150 having the digital telepathy app 122, and machine learning and artificial intelligence 170 devices that automatically captures and predicts a user's well-being 180. The computer 150 is used for sending the artificial intelligence user well-being determination information 182 and transmitting well-being indications 184 to the user smart phone 120. The user can view a display of the well-being determination information 127 and decide to select at least one person with whom the user wants to share the information 186. To assist the user and others with whom the user shares the determination information the ecosystem is providing emotional state-based intervention applications for a user and others to help users overcome troublesome emotions 190 of one embodiment.

### DETAILED DESCRIPTION

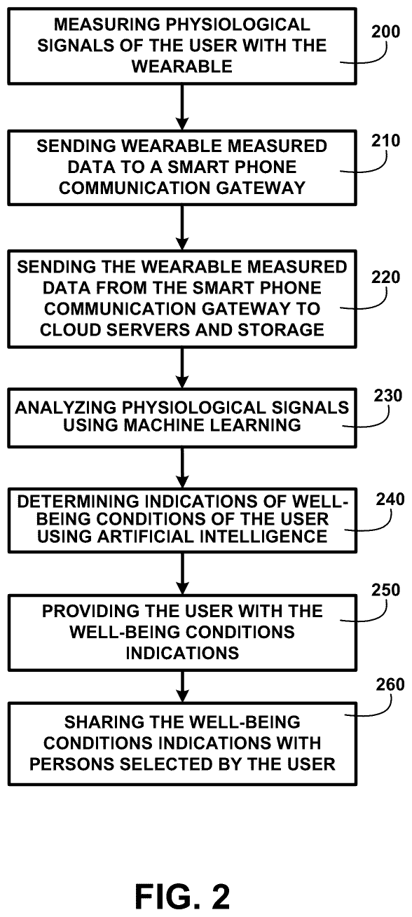

FIG. 2 shows a block diagram of an overview flow chart of a digital telepathy ecosystem of one embodiment. FIG. 2 shows a digital telepathy ecosystem measuring physiological signals of the user with the wearable 200 and sending wearable measured data to a smart phone communication gateway 210. Sending the wearable measured data from the smart phone communication gateway to cloud servers and storage 220 for additional processing. The processing includes analyzing physiological signals using machine learning 230 and determining indications of well-being conditions of the user using artificial intelligence 240. The physiological signals analysis is for providing the user with the well-being conditions indications 250 and sharing the well-being conditions indications with persons selected by the user 260 of one embodiment.

### A Digital Telepathy Ecosystem:

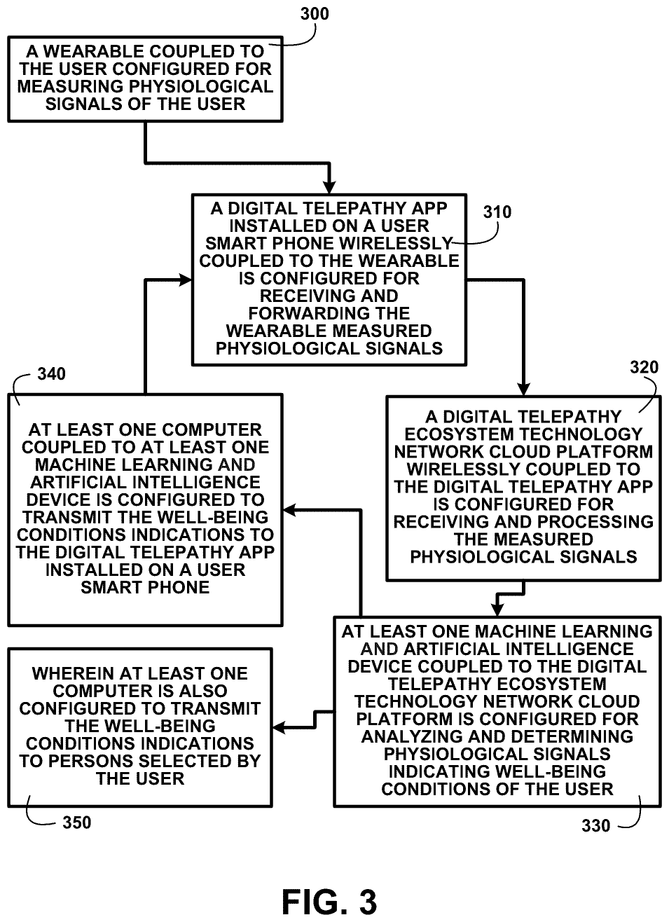

FIG. 3 shows a block diagram of an overview of a digital telepathy ecosystem of one embodiment. FIG. 3 shows a wearable coupled to the user configured for measuring physiological signals of the user 300. A digital telepathy app installed on a user smart phone wirelessly coupled to the wearable is configured for receiving and forwarding the wearable measured physiological signals 310. A digital telepathy ecosystem technology network cloud platform wirelessly coupled to the digital telepathy app is configured for receiving and processing the measured physiological signals 320. At least one machine learning and artificial intelligence device coupled to the digital telepathy ecosystem technology network cloud platform is configured for analyzing and determining physiological signals indicating the well-being conditions of the user 330. At least one computer coupled to at least one machine learning and artificial intelligence device is configured to transmit the well-being conditions indications to the digital telepathy app installed on a user smart phone 340, wherein at least one computer is also configured to transmit the well-being conditions indications to persons selected by the user 350 of one embodiment.

### A Digital Telepathy Ecosystem Technology Network Cloud Platform:

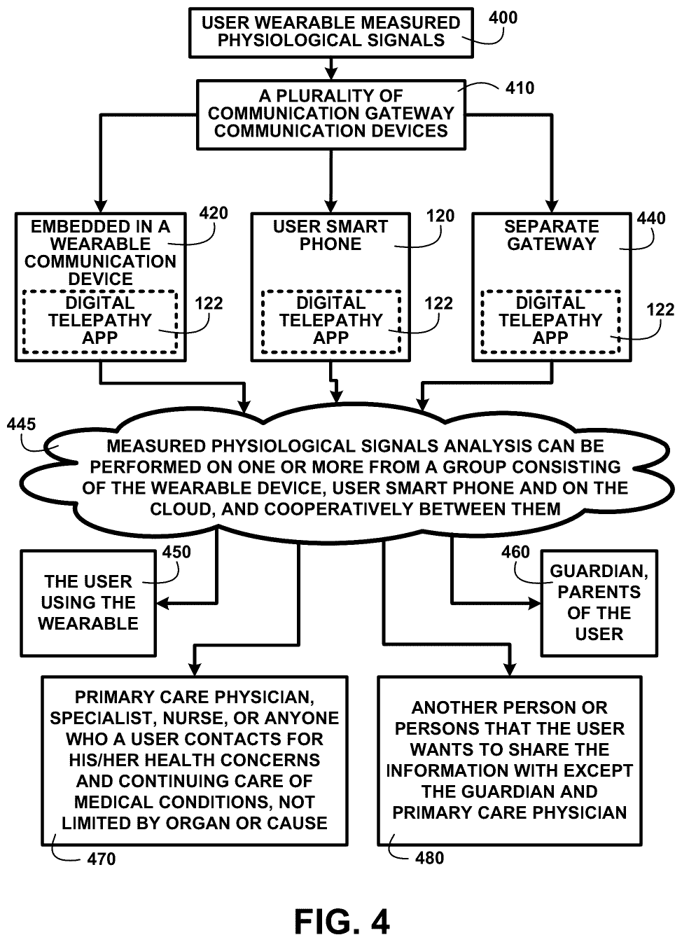

FIG. 4 shows for illustrative purposes only an example of a digital telepathy ecosystem technology network cloud platform of one embodiment. FIG. 4 shows user wearable measured physiological signals 400 that are transmitted using a plurality of communication gateway communication devices 410. The plurality of communication gateway communication devices 410 include, for example, a communication gateway embedded in a wearable communication device 420 having the digital telepathy app 122, a user smart phone 120 having the digital telepathy app 122, and a separate gateway 440 having the digital telepathy app 122.

Measured physiological signals analysis can be performed on one or more from a group consisting of the wearable device, user smart phone, and on the cloud, and cooperatively between them 445 is sent to the user using the wearable 450, guardian, parents of the user 460, the user primary care physician, specialist, nurse, or anyone who a user contacts for his/her health concerns and continuing care of medical conditions, not limited by organ or cause 470, and another person or persons that the user wants to share the information with except the guardian and primary care physician 480. Where the analysis is done depends on the complexity of the algorithm as well as the level of privacy needed. If high privacy is needed, the analysis and associated computation can be done on the wearable device or the phone, however, this limits options to simpler algorithms. If more complex algorithms are needed the data can be pushed to the cloud, however, this requires user data to leave the user's phone and this should be done very carefully using encryption methods to avoid violating user privacy through leakage of the user's data of one embodiment.

### A Wearable:

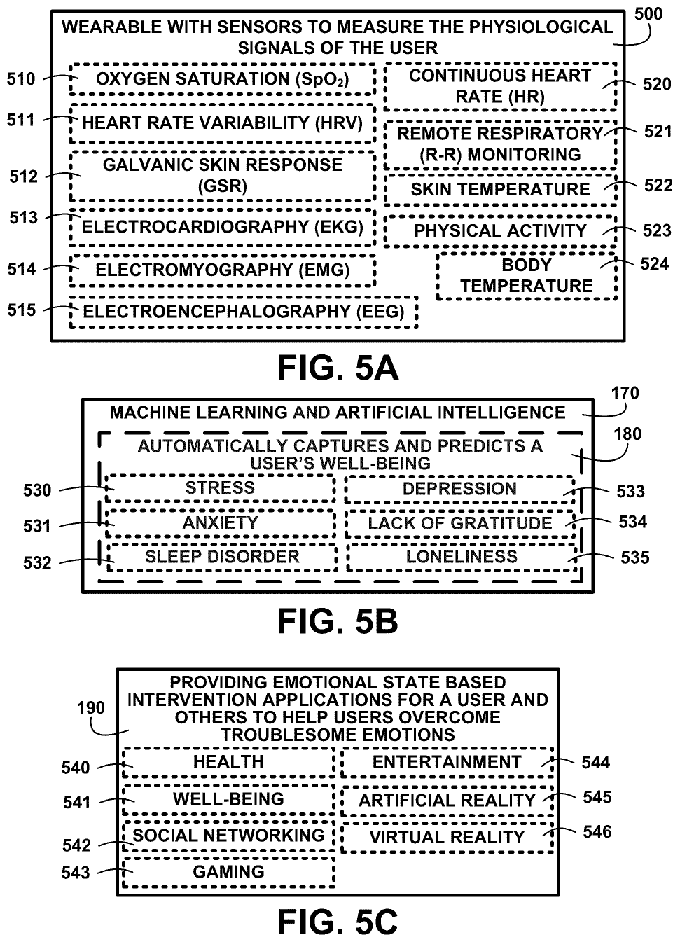

FIG. 5A shows a block diagram of an overview of a wearable of one embodiment. FIG. 5A shows a wearable with sensors to measure the physiological signals of the user 500 including oxygen saturation (SpO₂) 510, heart rate variability (HRV) 511, galvanic skin response (GSR) 512, electrocardiography (EKG) 513, electromyography (EMG) 514, electroencephalography (EEG) 515, continuous heart rate (HR) 520, remote respiratory (R-R) monitoring 521, skin temperature 522, physical activity 523, and body temperature 524 of one embodiment.

### Machine Learning and Artificial Intelligence:

FIG. 5B shows a block diagram of an overview of machine learning and artificial intelligence of one embodiment. FIG. 5B shows machine learning and artificial intelligence 170 devices that automatically capture and predicts a user's well-being 180. The machine learning analyses the user measured physiological signals compared to clinical symptoms of well-being conditions. It may run and be stored in the digital telepathy ecosystem technology network cloud platform 140 of FIG. 1, the wearable, the cellphone, or the gateway. The artificial intelligence correlates the user's activities during the measured physiological signals and the comparative well-being conditions to determine indications of the user's emotional state including stress 530, anxiety 531, sleep disorder 532, depression 533, lack of gratitude 534, and loneliness 535 of one embodiment.

### Intervention Applications:

FIG. 5C shows a block diagram of an overview of intervention applications of one embodiment. FIG. 5C shows providing emotional state-based intervention applications for a user and others to help users overcome troublesome emotions 190. The emotional state-based intervention applications can be integrated into applications related to health 540, well-being 541, social networking 542, gaming 543, entertainment 544, artificial reality 545, and virtual reality 546 of one embodiment.

### A Machine Learning Process:

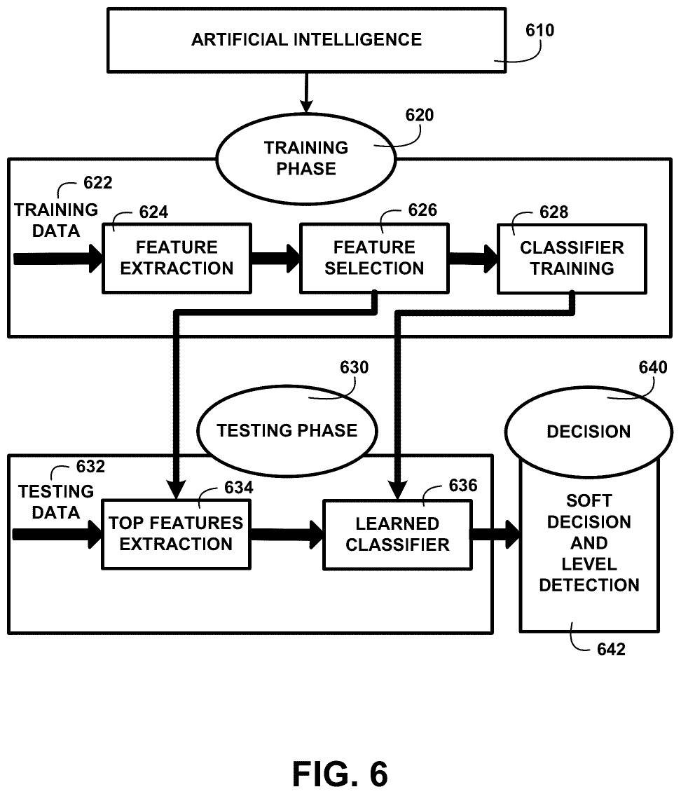

FIG. 6 shows for illustrative purposes only an example of a machine learning process of one embodiment. FIG. 6 shows the training of artificial intelligence 610 for determining emotional state indications. A training phase 620 uses training data 622 for feature extraction 624, feature selection 626, and classifier training 628. A testing phase 630 uses testing data 632 for evaluating top features extraction 634 and learned classifier 636 decision 640 results in soft decision and level detection 642 of one embodiment.

### A Detected/Predicted Emotion:

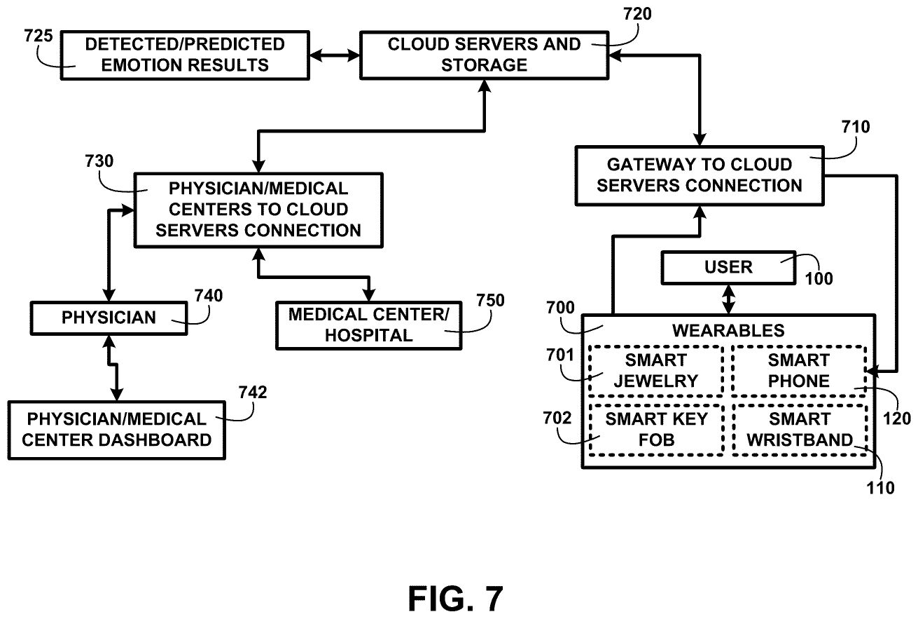

FIG. 7 shows a block diagram of an overview of a detected/predicted emotion of one embodiment. FIG. 7 shows the user 100 may select various wearables 700 apparatuses including, for example, a smart wristband 110, smart phone 120, smart jewelry 701, and a smart key fob 702 to send measured physiological signals to a gateway to cloud servers connection 710. This same connection can send information related to the measured physiological signals to a user smart phone. Cloud servers and storage 720 receive detected/predicted emotion results 725. The results can be sent to a physician/medical center to a cloud server connection 730 for use by a physician 740. The physician 740 can additionally review the information on a physician/medical center dashboard 742. This same connection can send information related to the measured physiological signals to a medical center/hospital 750 of one embodiment.

### Social Media Interactions:

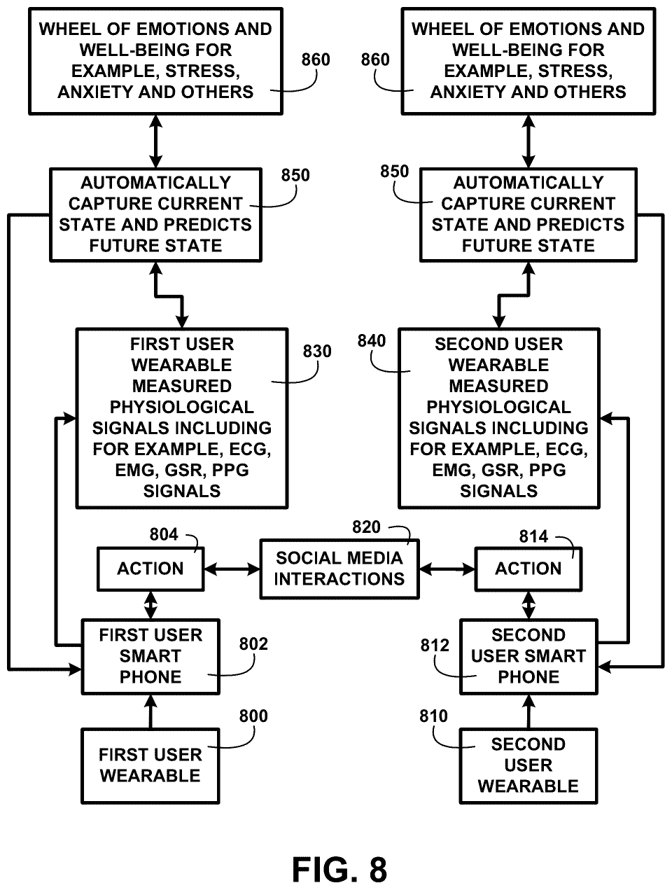

FIG. 8 shows a block diagram of an overview of social media interactions of one embodiment. FIG. 8 shows a first user wearable 800 sending data to a first user smart phone 802. The action 804 from the first user smart phone 802 includes the emotional state during social media interactions 820. A second user wearable 810 allows the second user via a second user smart phone 812 to review the action 814 of the first user social media interactions 820. The first user wearable measured physiological signals including, for example, ECG, EMG, GSR, and PPG signals 830 along with a few other signals from the phone such as audio, acceleration, and location that provides the context of the surrounding to automatically capture current state and predicts future state 850. A wheel of emotions and well-being, for example, stress, anxiety, and others 860 is displayed on the first user smart phone 802 to provide an understanding of the results. Second, user wearable measured physiological signals including for example ECG, EMG, GSR, and PPG signals 840 are also processed to automatically capture the current state and predicts future state 850, and the second user is also shown the wheel of emotions and well-being, for example, stress, anxiety and others 860 to provide an understanding of the emotions and well-being of one embodiment.

### Empathy in Metaverse:

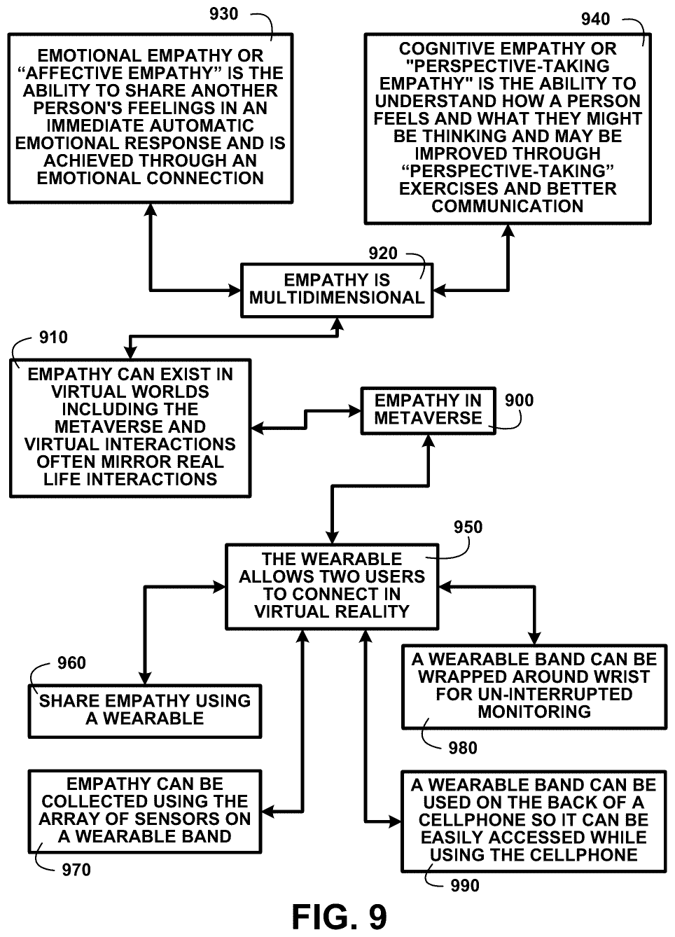

FIG. 9 shows a block diagram of an overview of empathy in the metaverse of one embodiment. FIG. 9 shows empathy in metaverse 900 to illustrate empathy can exist in virtual worlds including the metaverse and virtual interactions often mirror real-life interactions 910. Empathy is multidimensional 920 and includes emotional empathy or “affective empathy” which is the ability to share another person's feelings in an immediate automatic emotional response and is achieved through an emotional connection 930. Another dimension of empathy includes cognitive empathy, or “perspective-taking empathy” which is the ability to understand how a person feels and what they might be thinking and maybe improved through “perspective-taking” exercises and better communication 940. The wearable allows two users to connect in virtual reality 950 to share empathy using a wearable 960 along with emotional emoji/gifs in the chat environment. Empathy can be collected using the array of sensors on a wearable band 970. A wearable band can be wrapped around the wrist for uninterrupted monitoring 980. A wearable band can be used on the back of a cellphone so it can be easily accessed while using the cellphone 990 of one embodiment.

Empathy in metaverse can be used for various applications such as targeted advertisement, child monitoring, mental healthcare, and content curation to name a few. Wearable electronics make it possible to monitor human activity and behavior. In the future interconnected world where the computing systems are ubiquitous and interleaved in our daily life, evidence-based, scientific automatic capturing and prediction of human emotion and well-being using computing technology will be crucial to allow wearable technology to interact with us in an uninterrupted, non-disturbing, and non-disruptive manner for empathy sharing. Emotions as of today have not crossed the boundary of digital domains. Empowering the wearable technologies with advanced ML and AI to automatically capture and predict emotions will be the enabler of the next disruptive technology in human-computer interaction allowing many new applications in areas such as health, well-being, social networking, gaming, and entertainment, artificial reality, and virtual reality, just to name a few.

For instance, emotion can be used to create more strong bonds between individuals that are far away, and emotions can be used by various businesses to better serve the individuals based on their emotional states. By enabling the capture and sharing of information we can impact various fields such as social media, dating websites, video streaming services, entertainment/fashion industry, online gaming, advertisement industry, and telemedicine to name a few. We aim to create Embrace an ecosystem that allows continuous vital signal collection with an emotion detection algorithm that allows various entities to use emotions as one of the parameters for enabling and adding new dimensions to their solutions in the metaverse.

By capturing emotions using, we could establish empathy between various individuals and as the first step in introducing emotions in the virtual world, the first founding goal of the project is to improve the parent-child relationship using parental control platform called Teenverse. The teenverse is the first goal in creating the foundation for empathy sharing and establishing the solution. Health e-Tile technology enables parents to develop a better understanding of their kids' emotions. Teenverse is AI-enabled emotion understanding which correlates kid's emotions with an application being used on the handheld devices which gives parents more context on the behavior of their child.

### A Wearable Band on the Back of a Cellphone:

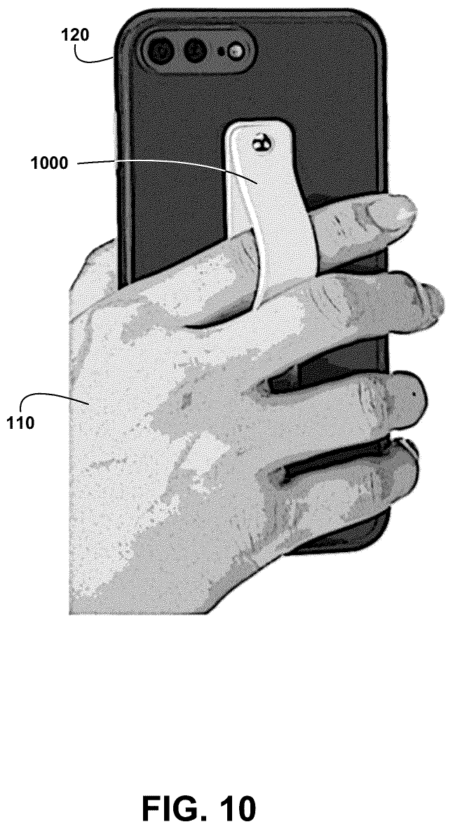

FIG. 10 shows for illustrative purposes only an example of a wearable band on the back of a cellphone of one embodiment. FIG. 10 shows the user 110 holding the user smart phone 120. The user 110 is in contact with a wearable band on the back of a cellphone 1000. The physical contact allows the wearable band on the back of a cellphone 1000 to collect and measure the physiological signals of one embodiment.

### Wearable Bands for Use by Two Persons:

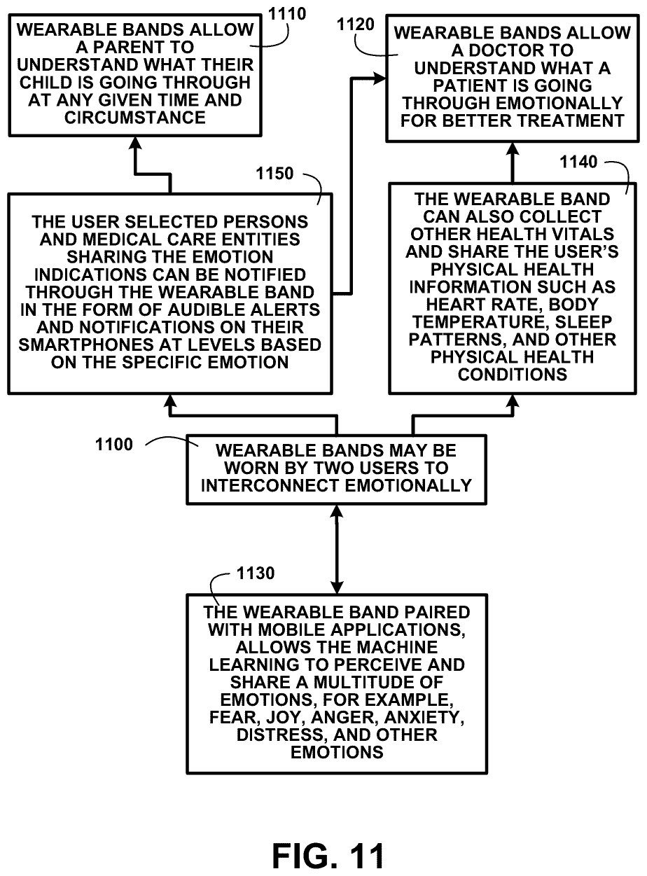

FIG. 11 shows a block diagram of an overview of wearable bands for use by two persons of one embodiment. FIG. 11 shows wearable bands may be worn by two users to interconnect emotionally 1100. For example, wearable bands allow a parent to understand what their child is going through at any given time and circumstance 1110. In another example, wearable bands allow a doctor to understand what a patient is going through emotionally for better treatment 1120. The wearable band paired with mobile applications, allows the machine learning to perceive and share a multitude of emotions, for example, fear, joy, anger, anxiety, distress, and other emotions 1130. The wearable band can also collect other health vitals and share the user's physical health information such as heart rate, body temperature, sleep patterns, and other physical health conditions 1140. The user-selected persons and medical care entities sharing the emotion indications can be notified through the wearable band in the form of audible alerts and notifications on their smartphones at levels based on the specific emotion 1150 to alert the selected persons and medical care entities of the user's emotional state and well-being conditions of one embodiment.

### Feeling the Emotional State in Real-Time:

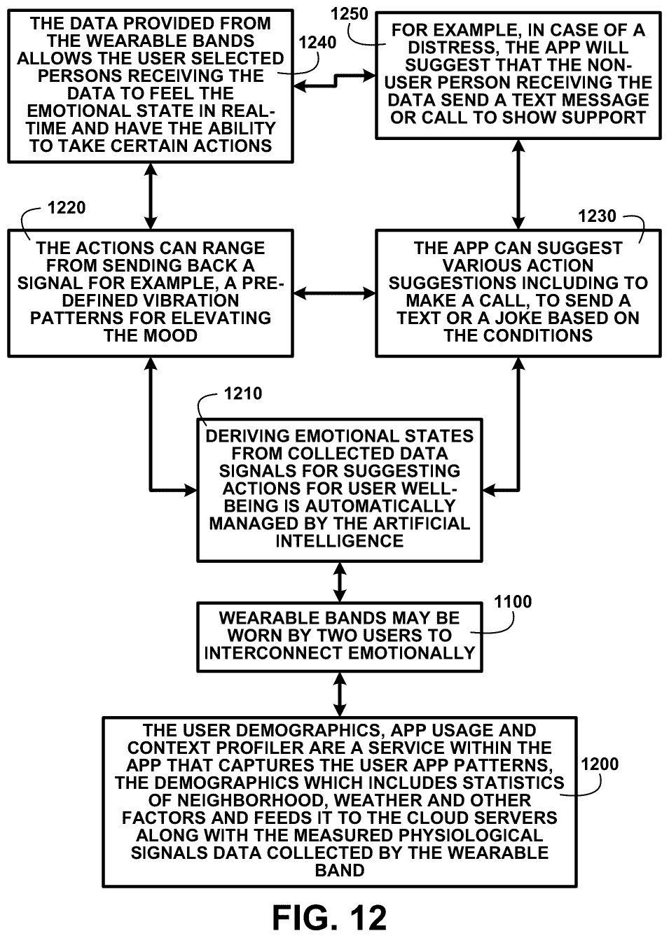

FIG. 12 shows a block diagram of an overview of feeling the emotional state in real-time of one embodiment. FIG. 12 shows wearable bands may be worn by two users to interconnect emotionally 1100. The user demographics, app usage, and context profiler are a service within the app that captures the user app patterns, the demographics which includes statistics of a neighborhood, weather, and other factors, and feeds it to the cloud servers along with the measured physiological signals data collected by the wearable band 1200. Deriving emotional states from collected data signals for suggesting actions for user well-being is automatically managed by the artificial intelligence 1210. The actions can range from sending back a signal, for example, pre-defined vibration patterns for elevating the mood 1220. The app can suggest various action suggestions including making a call, sending a text, or a joke based on the conditions 1230. The data provided from the wearable bands allows the user-selected persons receiving the data to feel the emotional state in real-time and have the ability to take certain actions 1240 for example, in case of distress, the app will suggest that the non-user person receiving the data send a text message or call to show support 1250 of one embodiment.

### Cognitive Empathy:

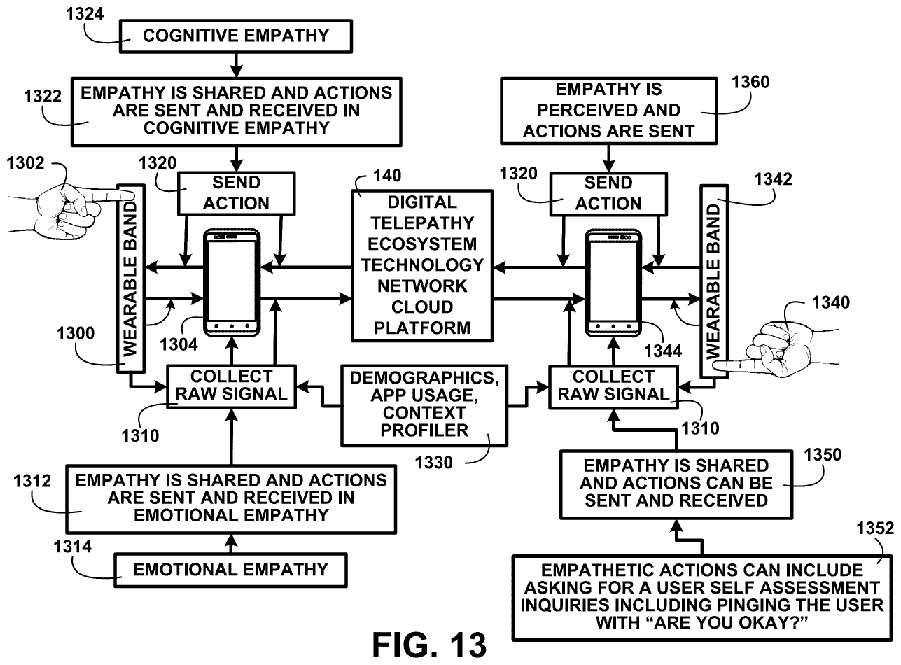

FIG. 13 shows for illustrative purposes only an example of cognitive empathy of one embodiment. FIG. 13 shows a first user wearable band 1300 in contact with a first user 1302 and wirelessly coupled to a first user smart phone 1304 to collect raw signal 1310. Empathy is shared and actions are sent and received in emotional empathy 1312. Emotional empathy 1314 can be a send action 1320 wherein empathy is shared, and actions are sent and received in cognitive empathy 1322. Cognitive empathy 1324 can in part be based on demographics, app usage, and context profiler 1330 provided through the digital telepathy ecosystem technology network cloud platform 140. A first user selected second user 1340 in contact with a second user wearable band 1342 can collect raw signal 1310 including demographics, app usage, context profiler 1330. Empathetic actions can include asking for a user self-assessment inquiries including pinging the user with “are you okay?” 1352. Empathy is shared and actions can be sent and received 1350 to collect raw signal 1310 on a second user smart phone 1344. Empathy is perceived and actions are sent 1360. The empathy send action 1320 is transmitted through the digital telepathy ecosystem technology network cloud platform 140 to the first user smart phone 1304 of one embodiment.

### Interactive Sexual Experiences:

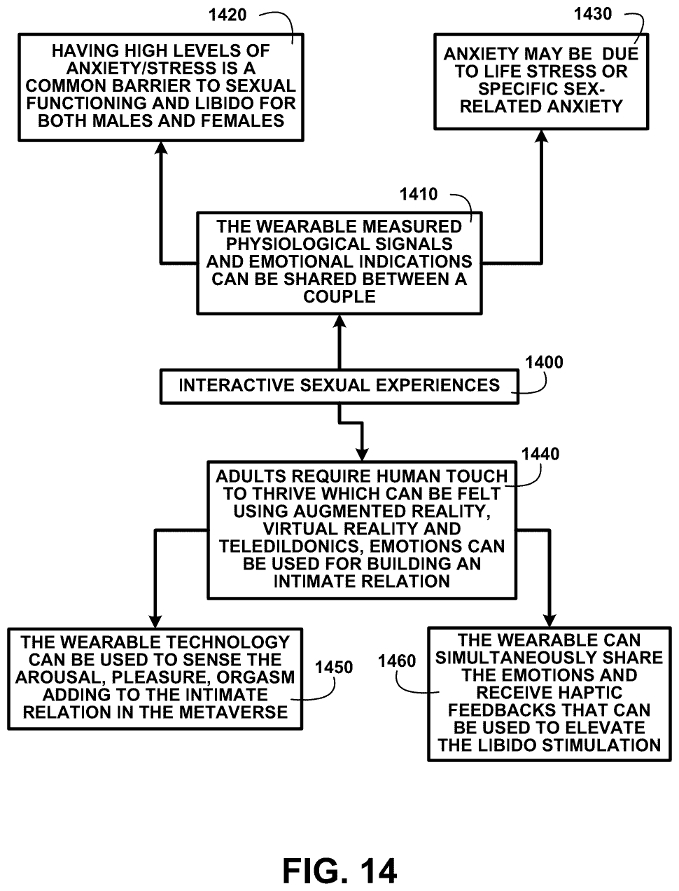

FIG. 14 shows a block diagram of an overview of interactive sexual experiences of one embodiment. FIG. 14 shows interactive sexual experiences 1400 wherein the wearable measured physiological signals and emotional indications can be shared between a couple 1410. Having high levels of anxiety/stress is a common barrier to sexual functioning and libido for both males and females 1420. Anxiety may be due to life stress or specific sex-related anxiety 1430. Adults require human touch to thrive which can be felt using augmented reality, virtual reality, and teledildonics, emotions can be used for building an intimate relation 1440. The wearable technology can be used to sense the arousal, pleasure, and orgasm adding to the intimate relation in the metaverse 1450. The wearable can simultaneously share the emotions and receive haptic feedback that can be used to elevate the libido stimulation 1460 of one embodiment.

### Cryptocurrency Rewards to Digital Telepathy Active Participants:

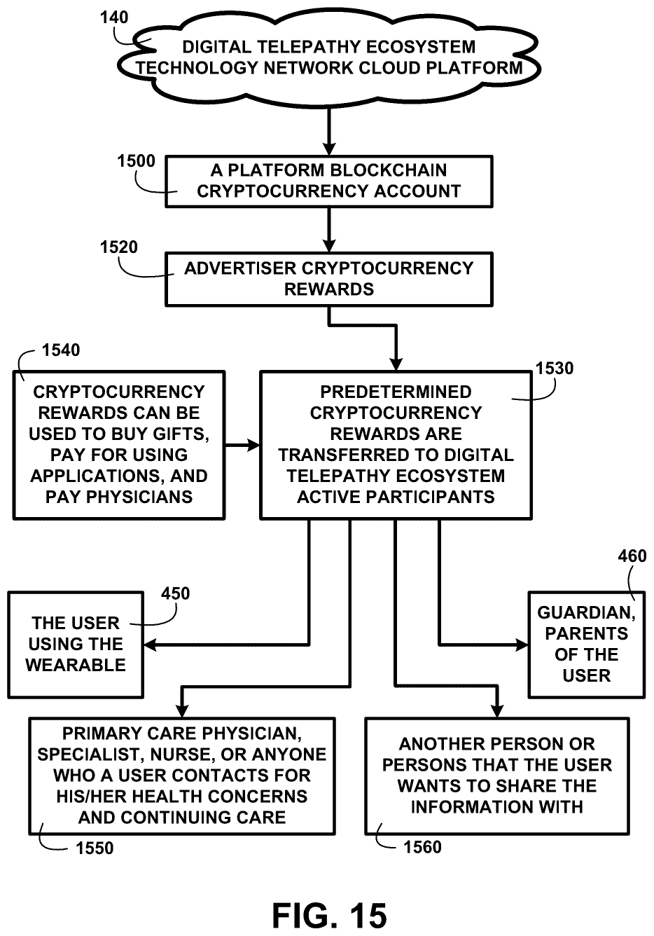

FIG. 15 shows for illustrative purposes only an example of cryptocurrency rewards to digital telepathy active participants of one embodiment. FIG. 15 shows the digital telepathy ecosystem technology network cloud platform 140 coupled to a platform blockchain cryptocurrency account 1500. The platform blockchain cryptocurrency account 1500 is used to receive advertiser cryptocurrency rewards 1520. Predetermined cryptocurrency rewards are transferred to digital telepathy ecosystem active participants 1530. Cryptocurrency rewards can be used to buy gifts, pay for using applications, and pay physicians 1540. The predetermined cryptocurrency rewards can be transferred to the user using the wearable 450, guardian, parents of the user 460, primary care physician, specialist, nurse, or anyone who a user contacts for his/her health concerns and continuing care 1550 and another person or persons that the user wants to share the information with 1560 of one embodiment.

### Advertising Products and Services Related to Targeted Emotional States:

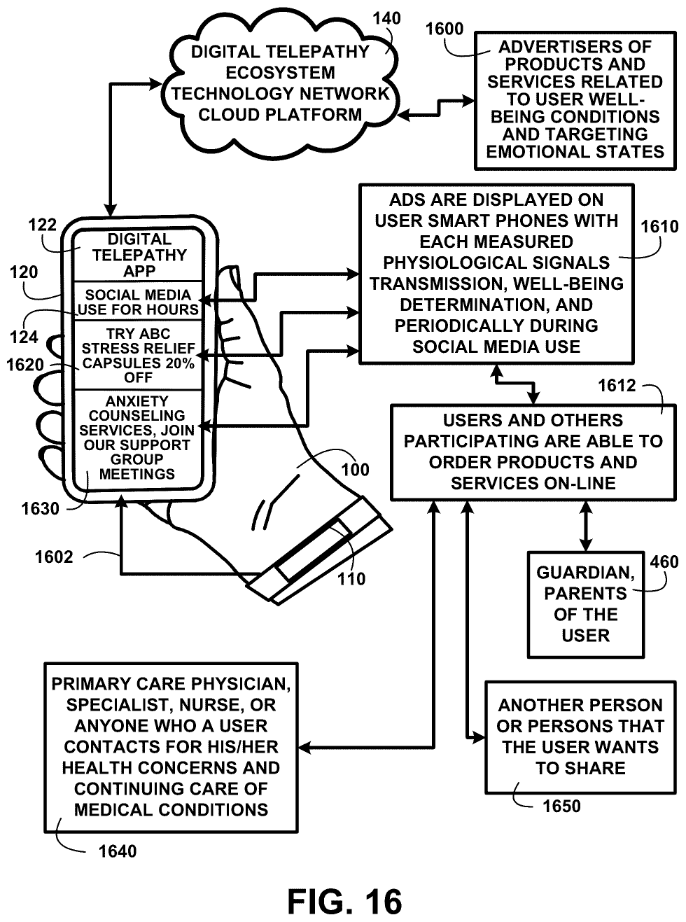

FIG. 16 shows for illustrative purposes only an example of advertising products and services related to targeted emotional states of one embodiment. FIG. 16 shows the user 100 in contact with the wearable 110 to send physiological signals to the user smart phone 1602. The digital telepathy ecosystem technology network cloud platform 140 is used to display ads of advertisers of products and services related to user well-being conditions and targeting emotional states 1600 on the user smart phone 120 with the digital telepathy app 122. The user uses social media for hours 124. Ads are displayed on the users' smart phones with each measured physiological signal transmission, well-being determination, and periodically during social media use 1610. Users and others participating are able to order products and services online 1612. Ads can be for example, “Try ABC stress relief capsules 20% off” 1620. Other ads can include “Anxiety counseling services, join our support group meetings” 1630. Ads are also displayed for primary care physicians, specialists, nurses, or anyone who a user contacts for his/her health concerns and continuing care of medical conditions 1640, guardian, parents of the user 460, and another person or persons that the user wants to share 1650 of one embodiment.

The emotional states can be used for targeting the right content, and advertisement. For example, it can be learned that a person becomes happy when the user buys things that they were eyeing and, in such conditions, target ads can be placed to make it easier for the user and the company to increase business. The emotional state can also be used to suggest media content. Clustering users with the same emotional state and recommending media content to the cluster. Patterns can also be discovered as to what users try to watch based on their emotional state.

### Selected Persons Provide a Level of Psychotherapy:

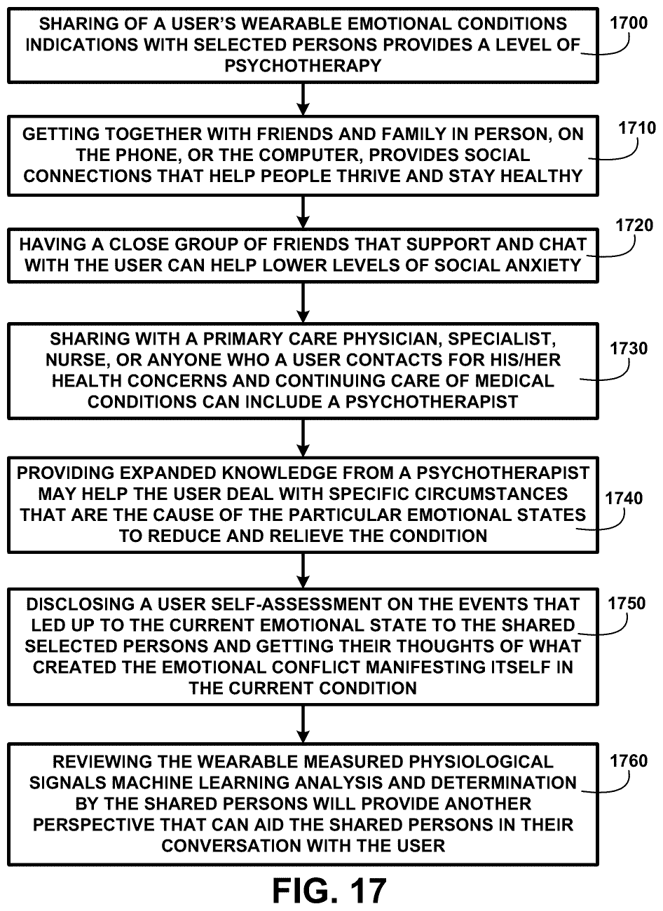

FIG. 17 shows a block diagram of an overview of selected persons providing a level of psychotherapy of one embodiment. FIG. 17 shows sharing of a user's wearable emotional conditions indications with selected persons provides a level of psychotherapy 1700. Getting together with friends and family in person, on the phone, or on the computer, provides social connections that help people thrive and stay healthy 1710. Also, having a close group of friends that support and chat with the user can help lower levels of social anxiety 1720. Sharing with a primary care physician, specialist, nurse, or anyone who a user contacts for his/her health concerns and continuing care of medical conditions can include a psychotherapist 1730.

In some circumstances providing expanded knowledge from a psychotherapist may help the user deal with specific circumstances that are the cause of the particular emotional states to reduce and relieve the condition 1740. Disclosing a user self-assessment on the events that led up to the current emotional state of the shared selected persons and getting their thoughts of what created the emotional conflict manifesting itself in the current condition 1750. Reviewing the wearable measured physiological signals machine learning analysis and determination by the shared persons will provide another perspective that can aid the shared persons in their conversation with the user 1760 of one embodiment.

### Monitoring Adolescence Stress and Anxiety:

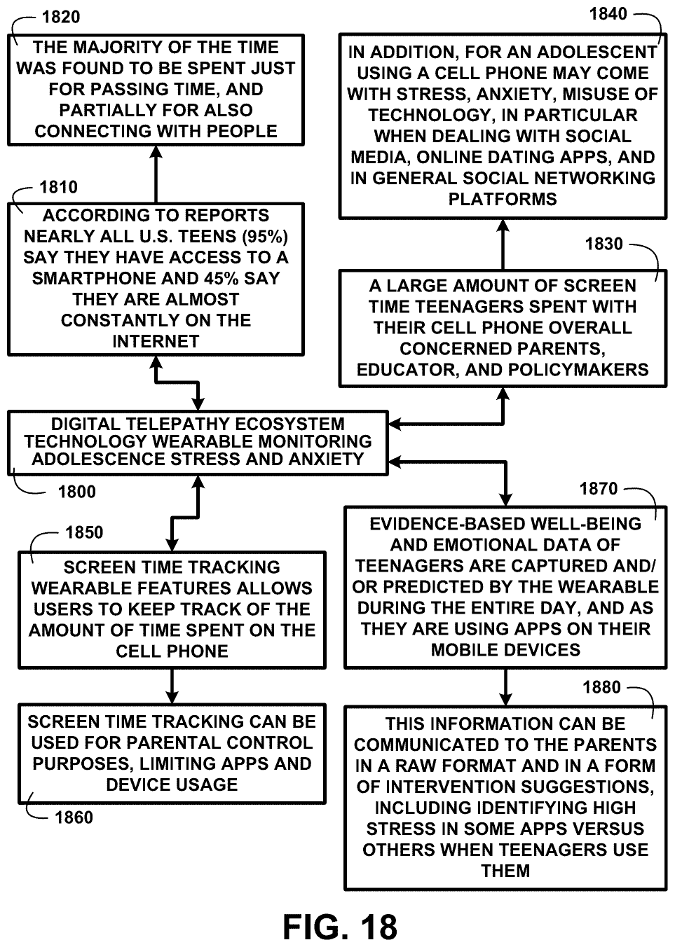

FIG. 18 shows a block diagram of an overview of monitoring adolescence stress and anxiety of one embodiment. FIG. 18 shows how digital telepathy ecosystem technology wearable monitoring adolescence stress and anxiety 1800 can prevent a continuation of adolescent emotional trauma. According to reports, nearly all U.S. teens (95%) say they have access to a smartphone and 45% say they are almost constantly on the internet 1810. The majority of the time was found to be spent just for passing time, and partially for also connecting with people 1820. A large amount of screen time teenagers spent with their cell phones overall concerned parents, educators, and policymakers 1830. In addition, for an adolescent using a cell phone may come with stress, anxiety, and misuse of technology, in particular when dealing with social media, online dating apps, and in general social networking platforms 1840. Screen time tracking wearable features allows users to keep track of the amount of time spent on the cell phone 1850. Screen time tracking can be used for parental control purposes, limiting apps and device usage 1860. Evidence-based well-being and emotional data of teenagers are captured and/or predicted by the wearable during the entire day and as they are using apps on their mobile devices 1870. This information can be communicated to the parents in a raw format and in a form of intervention suggestions, including identifying high stress in some apps versus others when teenagers use them 1880 of one embodiment.

### Evidence-Based Well-being and Emotion Data of Teenagers:

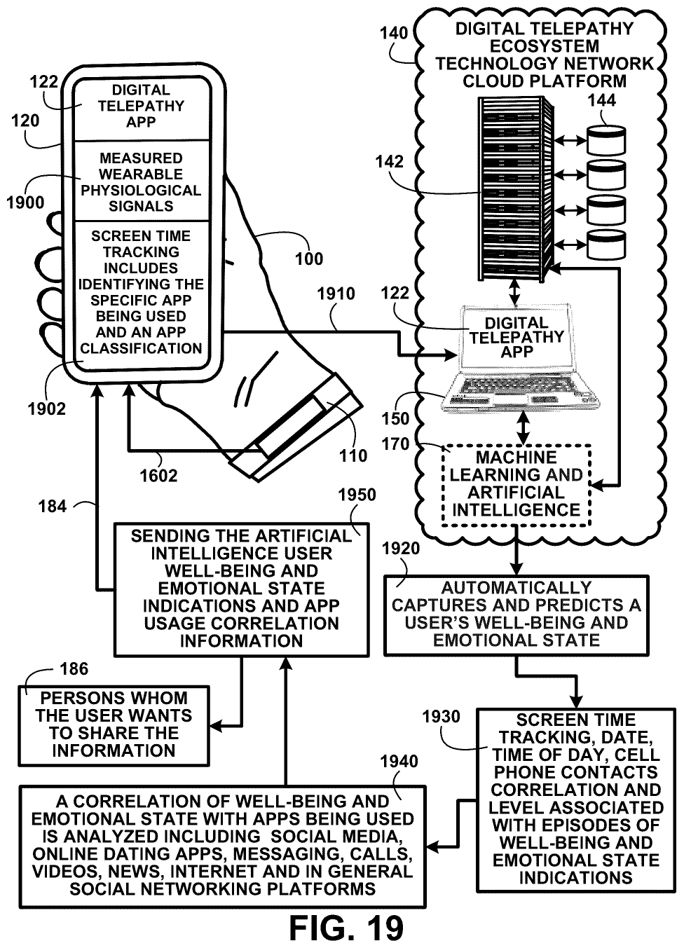

FIG. 19 shows a block diagram of an overview of evidence-based well-being and emotion data of teenagers of one embodiment. FIG. 19 shows the user 100 with a wearable 110 wristband. The wearable will send physiological signals to the user smart phone 1602. The user smart phone 120 has installed a digital telepathy app 122 configured for receiving measured wearable physiological signals 1900. Screen time tracking includes identifying the specific app being used and an app classification 1902 as social media, messaging, calls, videos, news, and the internet. The evidence-based well-being tracks the emotion based on the context of the app.

The user smart phone transmits 1910 measured wearable physiological signals and screen time tracking data to the digital telepathy ecosystem technology network cloud platform 140. At least one server 142 manages the data processing and at least one database 144 is used to store the raw data and processing results. The computer 150 receives the digital telepathy app 122 transmission of the measured wearable physiological signals and screen time tracking data. The machine learning and artificial intelligence 170 automatically captures and predicts a user's well-being and emotional state 1920. The screen time tracking, date, time of day, cell phone contacts correlation and level associated with episodes of well-being and emotional state indications 1930.

A correlation between well-being and emotional state with apps being used is analyzed including social media, online dating apps, messaging, calls, videos, news, the internet, and in general social networking platforms 1940. The computer 150 is used for sending the artificial intelligence user well-being and emotional state indications and app usage correlation information 1950 with transmitting well-being determination 184 to the user smart phone 120. In addition, the information is sent to persons with whom the user wants to share the information 186 of one embodiment.

### A Primary Care Physician Monitoring the Physiological Signals of a User:

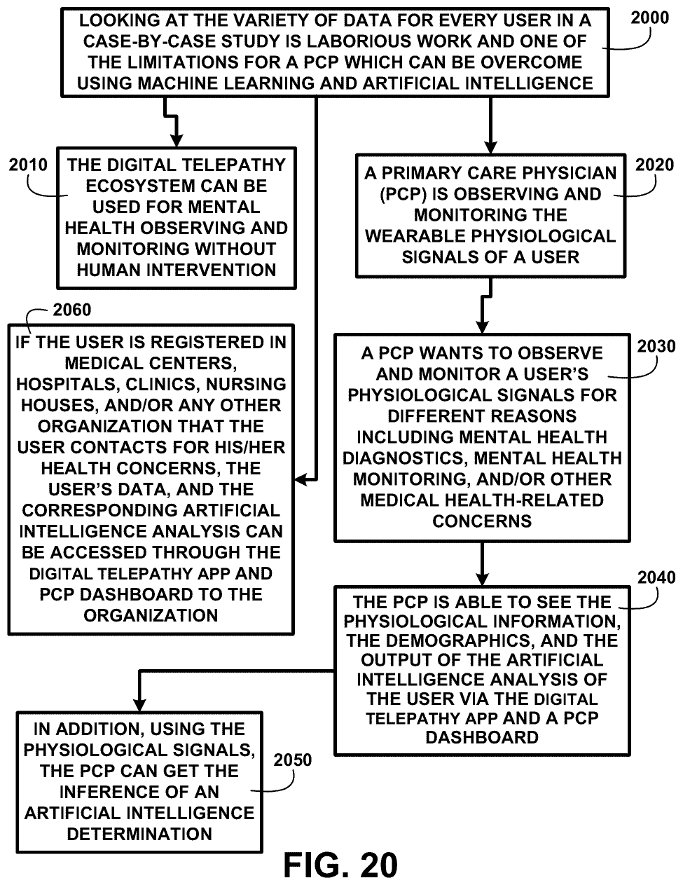

FIG. 20 shows a block diagram of an overview of a primary care physician monitoring the physiological signals of a user of one embodiment. FIG. 20 shows looking at the variety of data for every user in a case-by-case study is laborious work and one of the limitations for a PCP which can be overcome using machine learning and artificial intelligence 2000. The digital telepathy ecosystem can be used for mental health observing and monitoring without human intervention 2010. A primary care physician (PCP) is observing and monitors the wearable physiological signals of a user 2020. A PCP wants to observe and monitor a user's physiological signals for different reasons including mental health diagnostics, mental health monitoring, and/or other medical health-related concerns 2030.

The PCP is able to see the physiological information, the demographics, and the output of the artificial intelligence analysis of the user via the digital telepathy app and a PCP dashboard 2040. In addition, using the physiological signals, the PCP can get the inference of an artificial intelligence determination 2050. If the user is registered in medical centers, hospitals, clinics, nursing houses, and/or any other organization that the user contacts for his/her health concerns, the user's data, and the corresponding artificial intelligence analysis can be accessed through the digital telepathy app and PCP dashboard to the organization 2060 of one embodiment. This is one example of one too many types of communication i.e., PCP can communicate to many users.

### The User's Demographic Information:

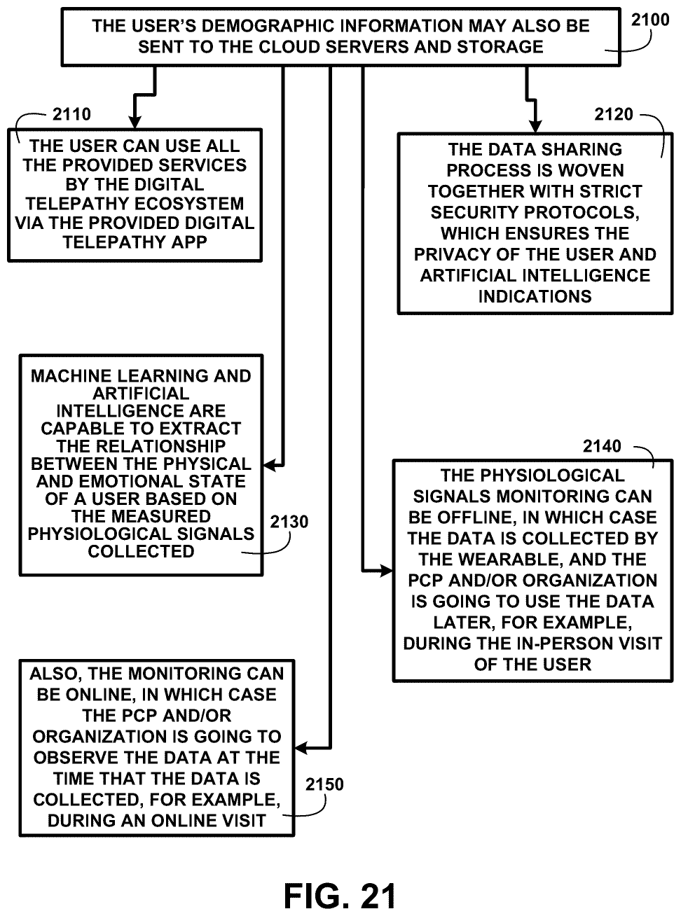

FIG. 21 shows a block diagram of an overview of the user's demographic information of one embodiment. FIG. 21 shows the user's demographic information may also be sent to the cloud servers and storage 2100. The user can use all the provided services by the digital telepathy ecosystem via the provided digital telepathy app 2110. The data sharing process is woven together with strict security protocols, which ensures the privacy of the user and artificial intelligence indications 2120. Machine learning and artificial intelligence are capable to extract the relationship between the physical and emotional state of a user based on the measured physiological signals collected 2130.

The physiological signals monitoring can be offline, in which case the data is collected by the wearable, and the PCP and/or organization is going to use the data later, for example, during the in-person visit of the user 2140. Also, the monitoring can be online, in which case the PCP and/or organization is going to observe the data at the time that the data is collected, for example, during an online visit 2150 one embodiment.

### Empowering Next Generation of Social Networking/Dating Apps:

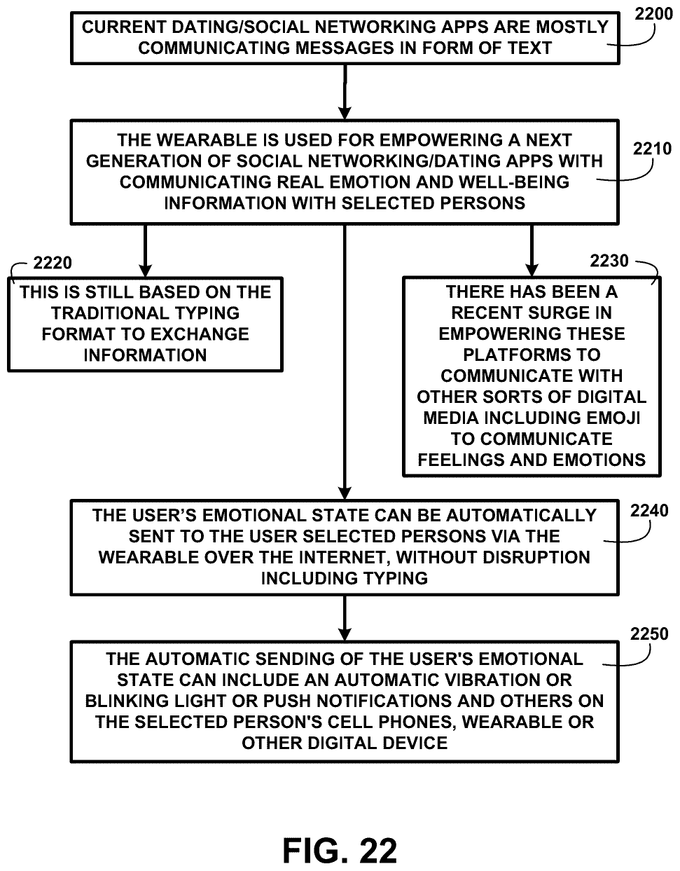

FIG. 22 shows a block diagram of an overview of empowering next generation of social networking/dating apps of one embodiment. FIG. 22 shows current dating/social networking apps are mostly communicating messages in form of text 2200. The wearable is used for empowering the next generation of social networking/dating apps with communicating real emotion and well-being information with selected persons 2210. This is still based on the traditional typing format to exchange information 2220.

There has been a recent surge in empowering these platforms to communicate with other sorts of digital media including emoji to communicate feelings and emotions 2230. The user's emotional state can be automatically sent to the user-selected persons via the wearable over the internet, without disruption including by typing 2240. The automatic sending of the user's emotional state can include an automatic vibration or blinking light or push notifications and others on the selected person's cell phones, wearable, or other digital devices 2250 of one embodiment.

Emotional States Integrated with an Emoji:

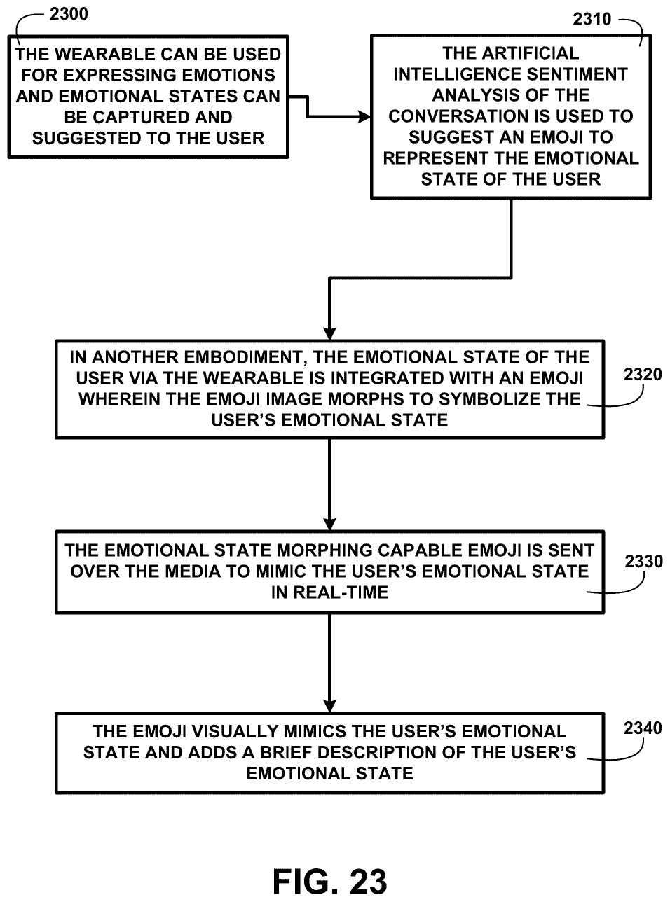

FIG. 23 shows a block diagram of an overview of emotional states integrated with an emoji of one embodiment. FIG. 23 shows the wearable can be used for expressing emotions and emotional states can be captured and suggested to the user 2300. The artificial intelligence sentiment analysis of the conversation is used to suggest an emoji to represent the emotional state of the user 2310.

In another embodiment, the emotional state of the user via the wearable is integrated with an emoji wherein the emoji image morphs to symbolize the user's emotional state 2320. The emotional state morphing capable emoji is sent over the media to mimic the user's emotional state in real-time 2330. The emoji visually mimics the user's emotional state and adds a brief description of the user's emotional state 2340 of one embodiment. Apart from the emoji, sending a gif/video can be generated using AI that captures and gives another person the same emotional state.

Sharing Well-being Conditions Indications with Persons Selected by the User:

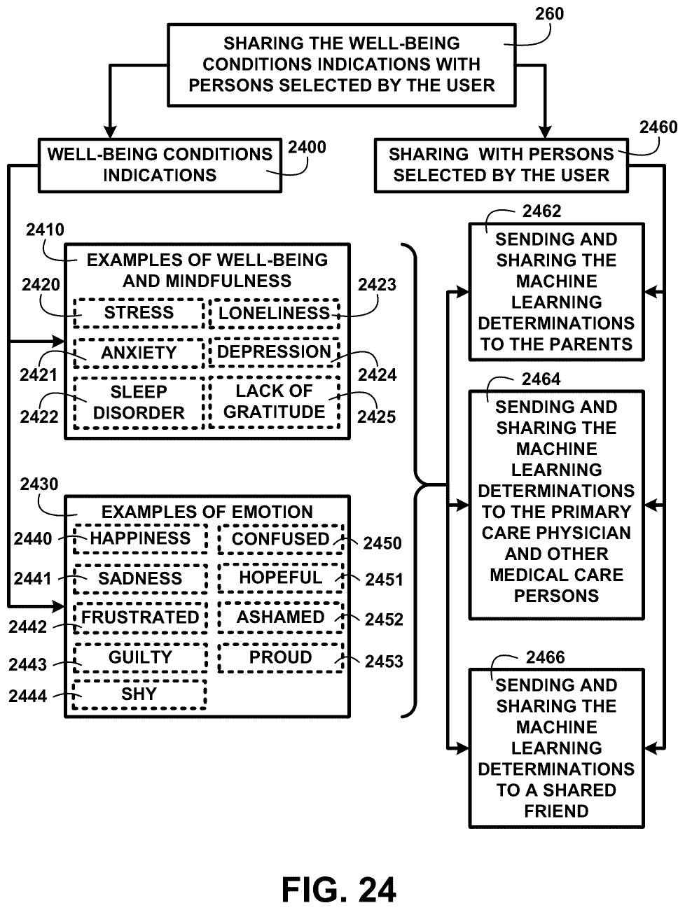

FIG. 24 shows a block diagram of an overview of sharing well-being conditions indications with persons selected by the user of one embodiment. FIG. 24 shows sharing the well-being conditions indications with persons selected by the user 260. The well-being conditions indications 2400 include examples of well-being and mindfulness 2410 for example, stress 2420, anxiety 2421, sleep disorder 2422, loneliness 2423, depression 2424, and lack of gratitude 2425.

The well-being conditions indications 2400 include examples of emotion 2430 for example, happiness 2440, sadness 2441, frustrated 2442, guilty 2443, shy 2444, confused 2450, hopeful 2451, ashamed 2452, and proud 2453. The digital telepathy app 122 of FIG. 1 allows sharing with persons selected by the user 2460. Sending and sharing the machine learning determinations to the parents 2462, sending and sharing the machine learning determinations to the primary care physician and other medical care persons 2464, and sending and sharing the machine learning determinations to a shared friend 2466 of one embodiment

### Application Integrated Intervention:

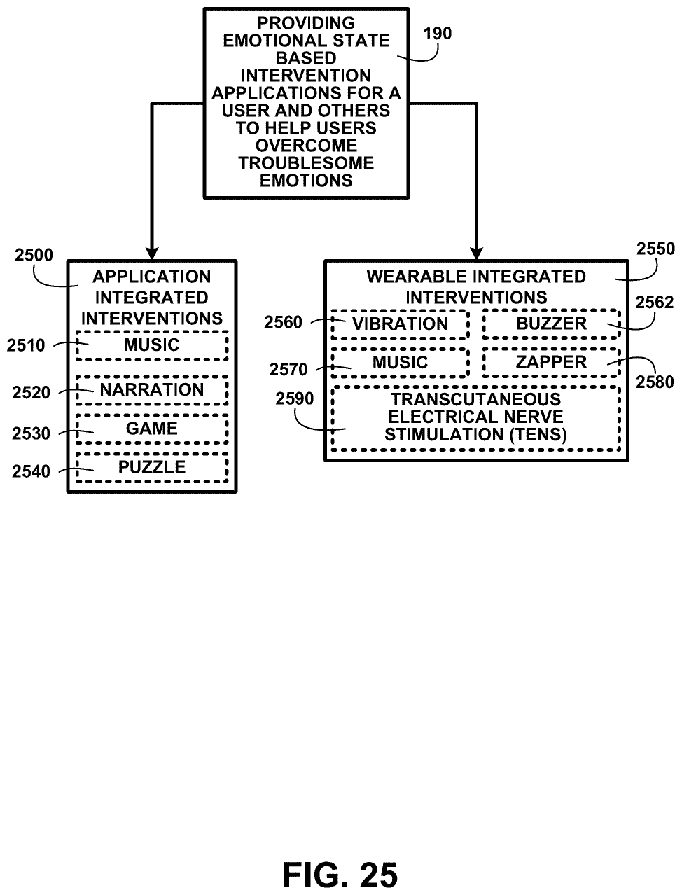

FIG. 25 shows a block diagram of an overview of application integrated intervention of one embodiment. FIG. 25 shows providing emotional state-based intervention applications for a user and others to help users overcome troublesome emotions 190. Interventions can assist the user in dealing with emotional states. Application integrated interventions 2500 include music 2510, narration 2520, a game 2530, and a puzzle 2540. These may distract the user from becoming overwhelmed by the emotions. Wearable integrated interventions 2550 can include for example, vibration 2560, a buzzer 2562, music 2570, a zapper 2580 and transcutaneous electrical nerve stimulation (TENS) 2590 of one embodiment.

### Artificial Intelligence:

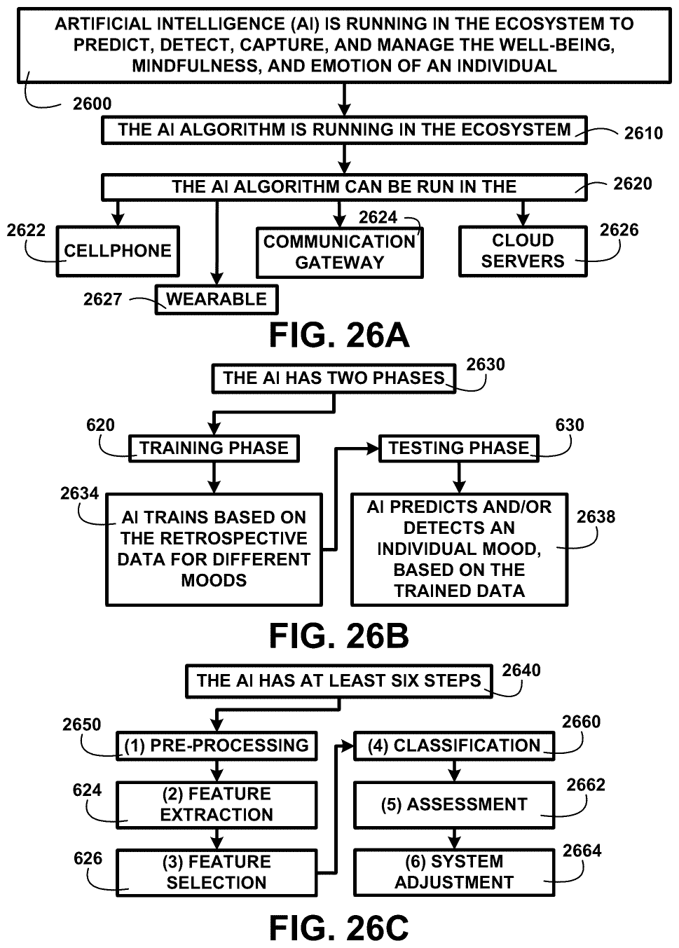

FIG. 26A shows a block diagram of an overview of artificial intelligence of one embodiment. FIG. 26A shows artificial intelligence (AI) is running in the ecosystem to predict, detect, capture, and manage the well-being, mindfulness, and emotion of an individual 2600. The AI algorithm is running in the ecosystem 2610. The AI algorithm can be run in the 2620 cellphone 2622, communication gateway 2624, wearable 2627, and the cloud servers 2626 of one embodiment.

### AI Two Phases:

FIG. 26B shows a block diagram of an overview of AI in two phases of one embodiment. FIG. 26B shows the AI has two phases 2630. The first phase is a training phase 620 that is followed by a second phase of a testing phase 630. AI trains based on the retrospective data for different moods 2634. AI predicts and/or detects an individual mood, based on the trained data 2638 of one embodiment.

### AI Six Steps:

FIG. 26C shows a block diagram of an overview of AI in six steps of one embodiment. FIG. 26C shows the AI has at least six steps 2640 including (1) pre-processing 2650, (2) feature extraction 624, (3) feature selection 626, (4) classification 2660, (5) assessment 2662, and (6) system adjustment 2664 of one embodiment.

### Pre-Processing:

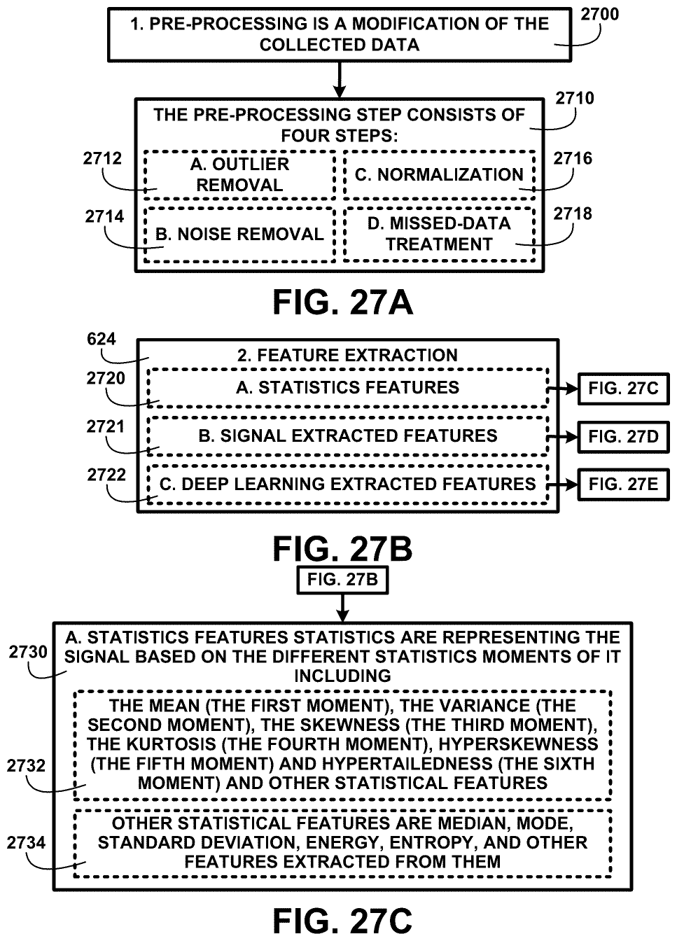

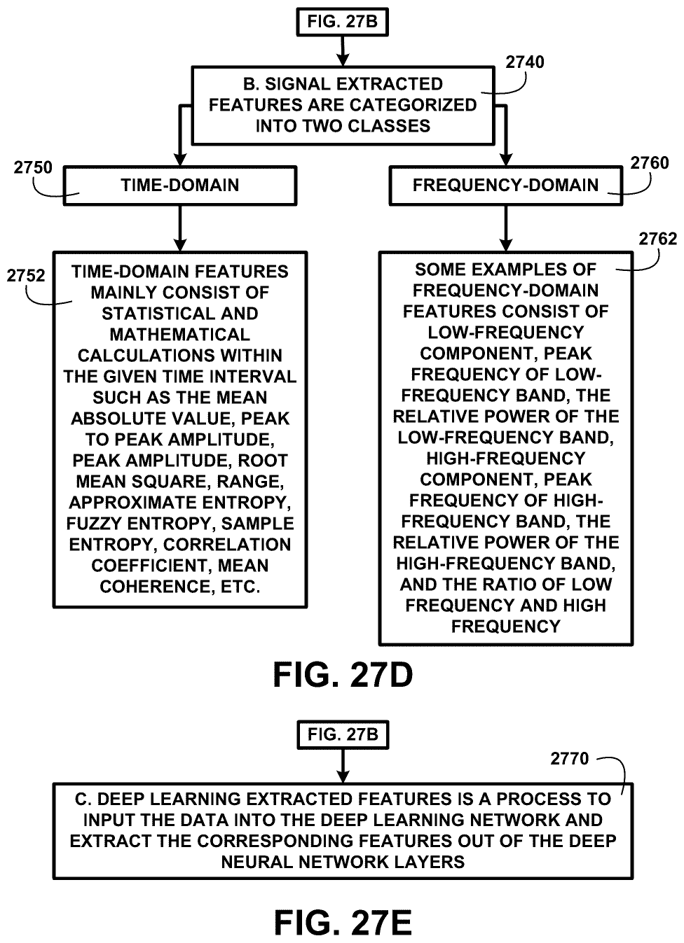

FIG. 27A shows a block diagram of an overview of pre-processing of one embodiment. FIG. 27A shows 1. Pre-processing is a modification of the collected data 2700. The pre-processing step consists of four steps: 2710. The four steps include a. outlier removal 2712, b. noise removal 2714, c. normalization 2716, and d. missed-data treatment 2718 of one embodiment.

### Feature Extraction:

FIG. 27B shows a block diagram of an overview of feature extraction of one embodiment. FIG. 27B shows 2. feature extraction 624 consisting of a. statistics features 2720 that is described in FIG. 27C; b. signal extracted features 2721 described in FIG. 27D; and c. deep learning extracted features 2722 described in FIG. 27E of one embodiment.

### Statistics Features:

FIG. 27C shows a block diagram of an overview of statistics features of one embodiment. FIG. 27C shows a continuation from FIG. 27B of a. statistics features 2720 of FIG. 27B. a. statistics features statistics are representing the signal based on the different statistics moments of it including 2730. The mean (the first moment), the variance (the second moment), the skewness (the third moment), the kurtosis (the fourth moment), hyperskewness (the fifth moment), and hypertailedness (the sixth moment) and other statistical features 2732. Other statistical features are median, mode, standard deviation, energy, entropy, and other features extracted from them 2734 of one embodiment.

### Signal Extracted Features:

FIG. 27D shows a block diagram of an overview of signal extracted features of one embodiment. FIG. 27D shows a continuation from FIG. 27B of b. signal extracted features 2721 of FIG. 27B. The b. signal extracted features are categorized into two classes 2740. The first class is time-domain 2750. Time-domain features mainly consist of statistical and mathematical calculations within the given time interval such as the mean absolute value, peak to peak amplitude, peak amplitude, root mean square, range, approximate entropy, fuzzy entropy, sample entropy, correlation coefficient, and mean coherence, etc. 2752. The second class is frequency-domain 2760. Some examples of frequency-domain features consist of low-frequency component, peak frequency of low-frequency band, the relative power of the low-frequency band, high-frequency component, peak frequency of high-frequency band, the relative power of the high-frequency band, and the ratio of low frequency and high frequency 2762 of one embodiment.

### Deep Learning Extracted Features:

FIG. 27E shows a block diagram of an overview of deep learning extracted features of one embodiment. FIG. 27E shows a continuation from FIG. 27B of c. deep learning extracted features 2722 of FIG. 27B. c. deep learning extracted features is a process to input the data into the deep learning network and extract the corresponding features out of the deep neural network layers 2770 of one embodiment.

### Feature Selection:

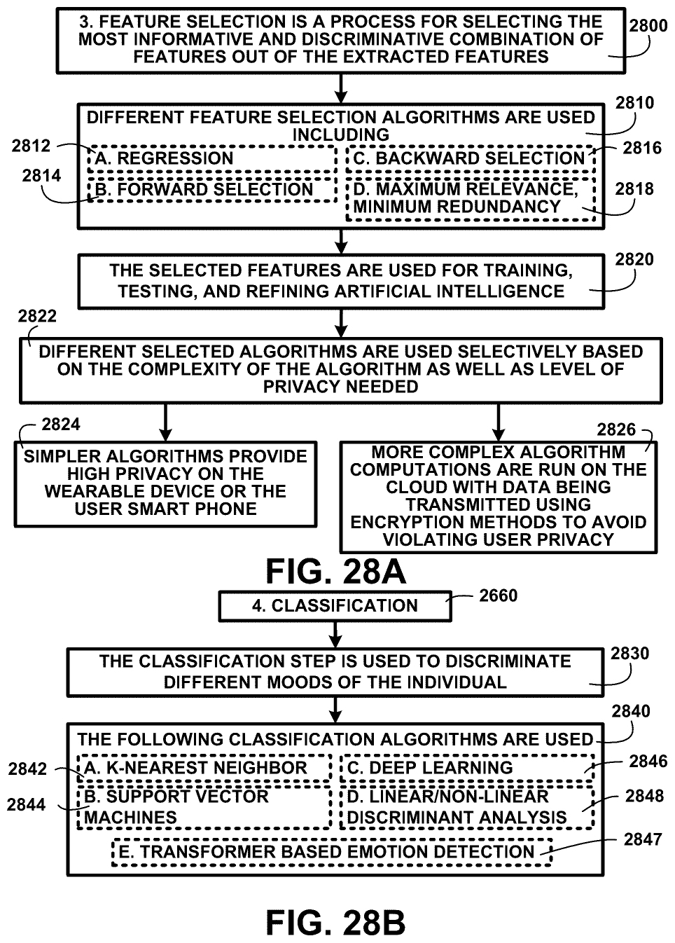

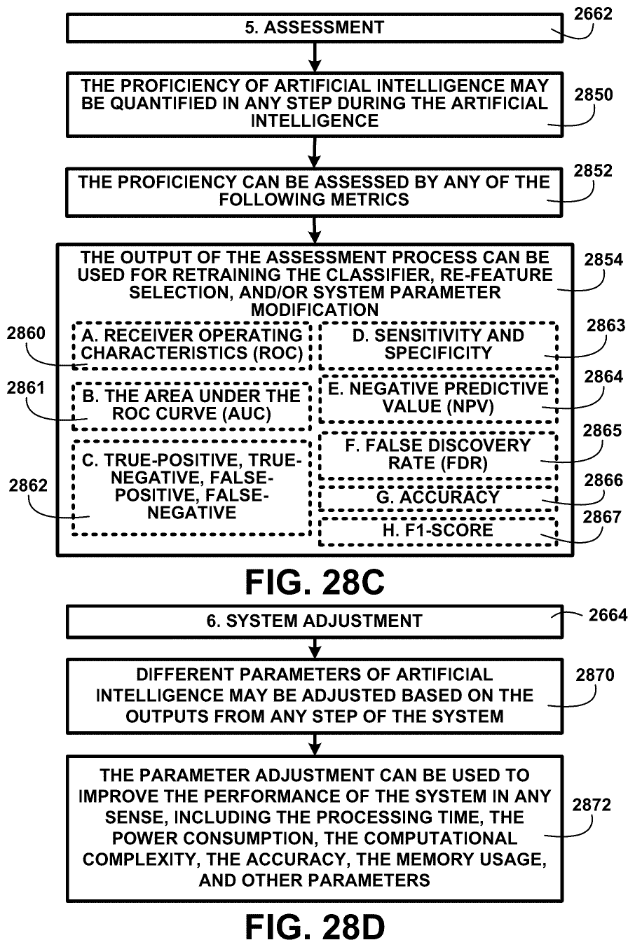

FIG. 28A shows a block diagram of an overview of feature selection of one embodiment. FIG. 28A shows 3. feature selection is a process for selecting the most informative and discriminative combination of features out of the extracted features 2800. Different feature selection algorithms are used including 2810 a. regression 2812, b. forward selection 2814, c. backward selection 2816, and d. maximum relevance, minimum redundancy 2818. The selected features are used for training, testing, and refining artificial intelligence 2820. Different selected algorithms are used selectively based on the complexity of the algorithm as well as the level of privacy needed 2822. Simpler algorithms provide high privacy on the wearable device or the user smart phone 2824. More complex algorithm computations are run on the cloud with data being transmitted using encryption methods to avoid violating user privacy 2826 of one embodiment.

### Classification:

FIG. 28B shows a block diagram of an overview of classification of one embodiment. FIG. 28B shows 4. classification 2660 where the classification step is used to discriminate different moods of the individual 2830. The following classification algorithms are used 2840 a. K-nearest neighbor 2842, b. support vector machines 2844, c. deep learning 2846, d. linear/non-linear discriminant analysis 2848, and e. transformer-based emotion detection 2847 of one embodiment.

### Assessment:

FIG. 28C shows a block diagram of an overview of assessment of one embodiment. FIG. 28C shows 5. assessment 2662 where the proficiency of artificial intelligence may be quantified in any step during the artificial intelligence 2850. The proficiency can be assessed by any of the following metrics 2852. The output of the assessment process can be used for retraining the classifier, re-feature selection, and/or system parameter modification 2854. Assessment metrics include a. receiver operating characteristics (ROC) 2860, b. the area under the roc curve (AUC) 2861, c. true-positive, true-negative, false-positive, false-negative 2862, d. sensitivity and specificity 2863, e. negative predictive value (NPV) 2864, f. false discovery rate (FDR) 2865, g. accuracy 2866, and h. F1-score 2867 of one embodiment.

### System Adjustment:

FIG. 28D shows a block diagram of an overview of system adjustment of one embodiment. FIG. 28D shows 6. system adjustment 2664 where different parameters of artificial intelligence may be adjusted based on the outputs from any step of the system 2870. The parameter adjustment can be used to improve the performance of the system in any sense, including the processing time, the power consumption, the computational complexity, the accuracy, the memory usage, and other parameters 2872 of one embodiment.

### Collecting User Emotion:

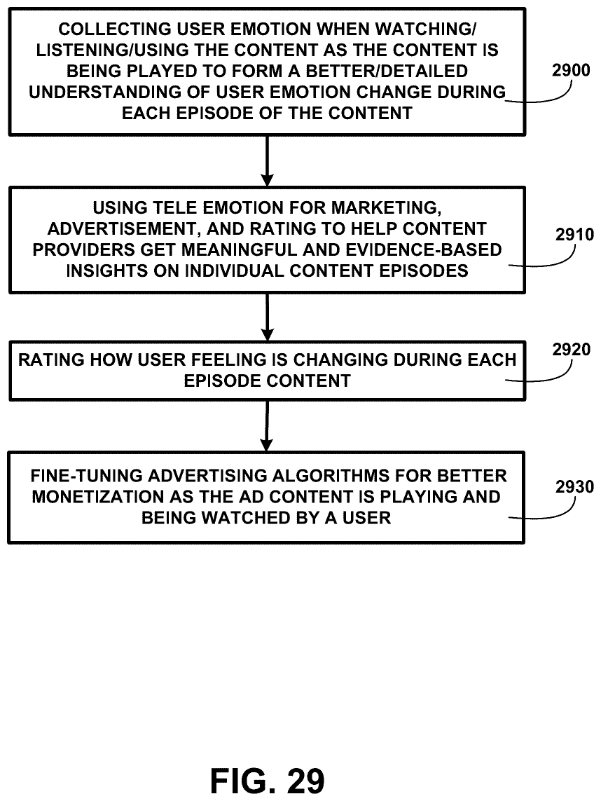

FIG. 29 shows a block diagram of an overview of collecting user emotion of one embodiment. FIG. 29 shows collecting user emotion when watching/listening/using the content as the content is being played to form a better/detailed understanding of user emotion change during each episode of the content 2900. Using tele-emotion for marketing, advertisement, and rating to help content providers get meaningful and evidence-based insights on individual content episodes 2910. Rating how user feeling is changing during each episode content 2920. Fine-tuning advertising algorithms for better monetization as the ad content is playing and being watched by a user 2930 of one embodiment. This is an example of one entity to many user situations for communication.

### Blockchain Technology:

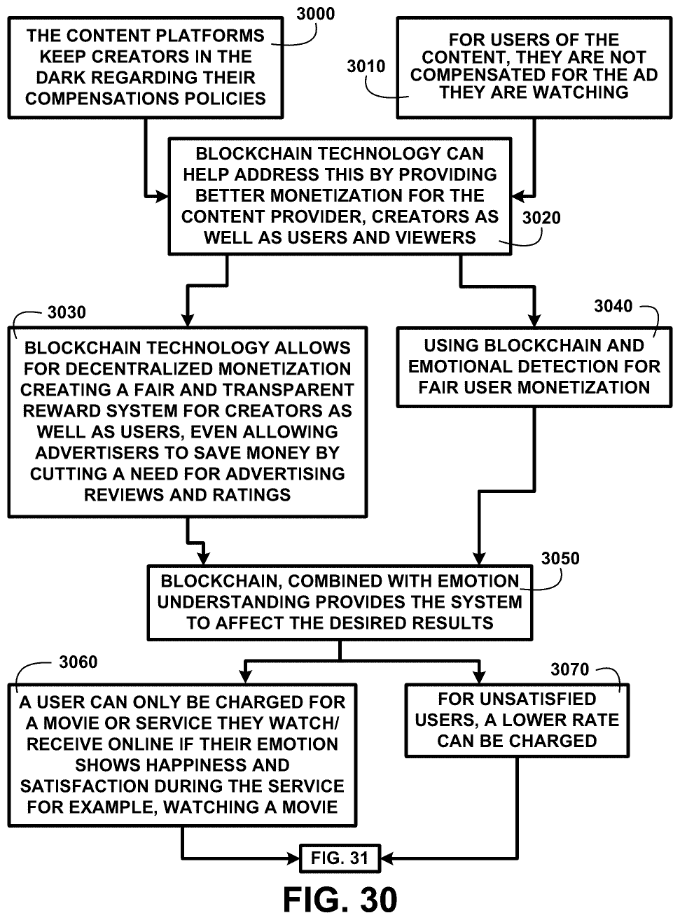

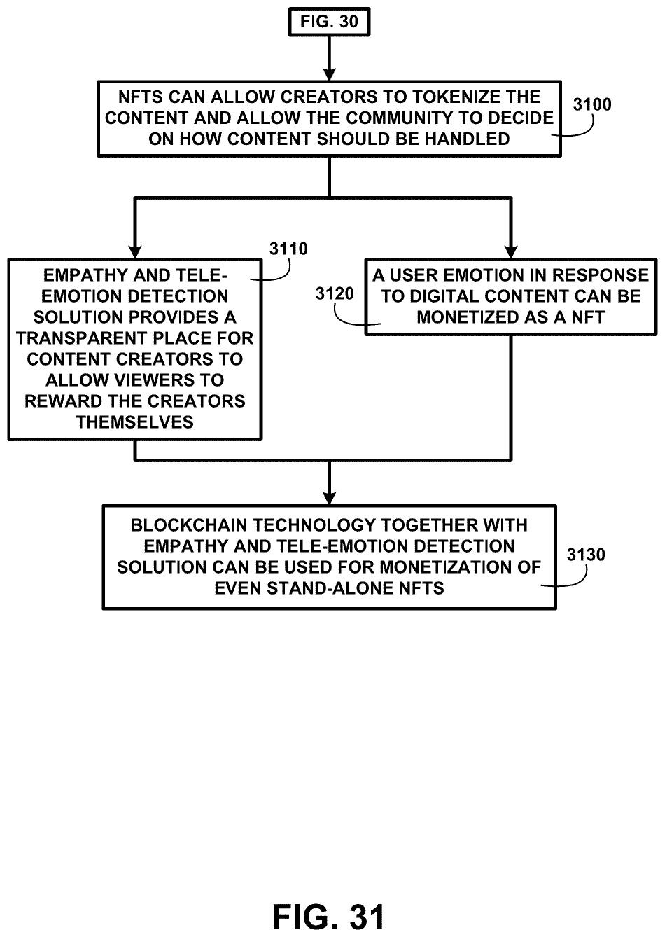

FIG. 30 shows a block diagram of an overview of blockchain technology of one embodiment. FIG. 30 shows the content platforms keep creators in the dark regarding their compensations policies 3000. For users of the content, they are not compensated for the ad they are watching 3010. Blockchain technology can help address this by providing better monetization for the content provider, creators as well as users and viewers 3020. Blockchain technology allows for decentralized monetization creating a fair and transparent reward system for creators as well as users, even allowing advertisers to save money by cutting the need for advertising reviews and ratings 3030. Using blockchain and emotional detection for fair user monetization 3040. Blockchain, combined with emotion understanding provides the system to affect the desired results 3050. A user can only be charged for a movie or service they watch/receive online if their emotion shows happiness and satisfaction during the service for example, watching a movie 3060. For unsatisfied users, a lower rate can be charged 3070 of one embodiment. The description continues in FIG. 31.

### NFTs:

FIG. 31 shows a block diagram of an overview of NFTs of one embodiment. FIG. 31 shows a continuation from FIG. 30. NFTs can allow creators to tokenize the content and allow the community to decide on how the content should be handled 3100. Empathy and tele-emotion detection solution provides a transparent place for content creators to allow viewers to reward the creators themselves 3110. A user emotion in response to digital content can be monetized as an NFT 3120. Blockchain technology together with empathy and tele-emotion detection solution can be used for monetization of even stand-alone NFTs 3130 of one embodiment.

### Framework for Emotional Empathy:

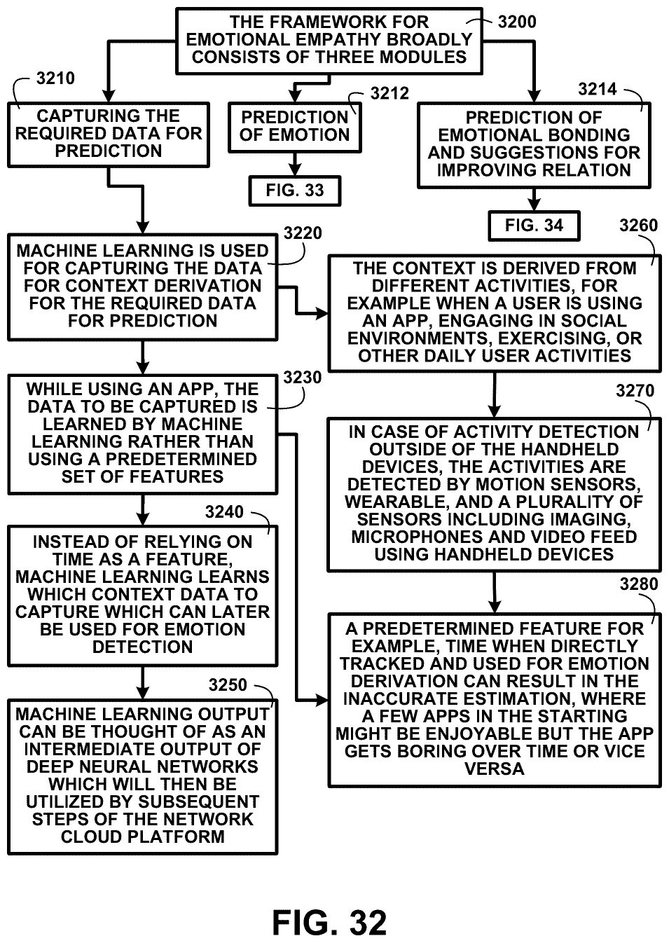

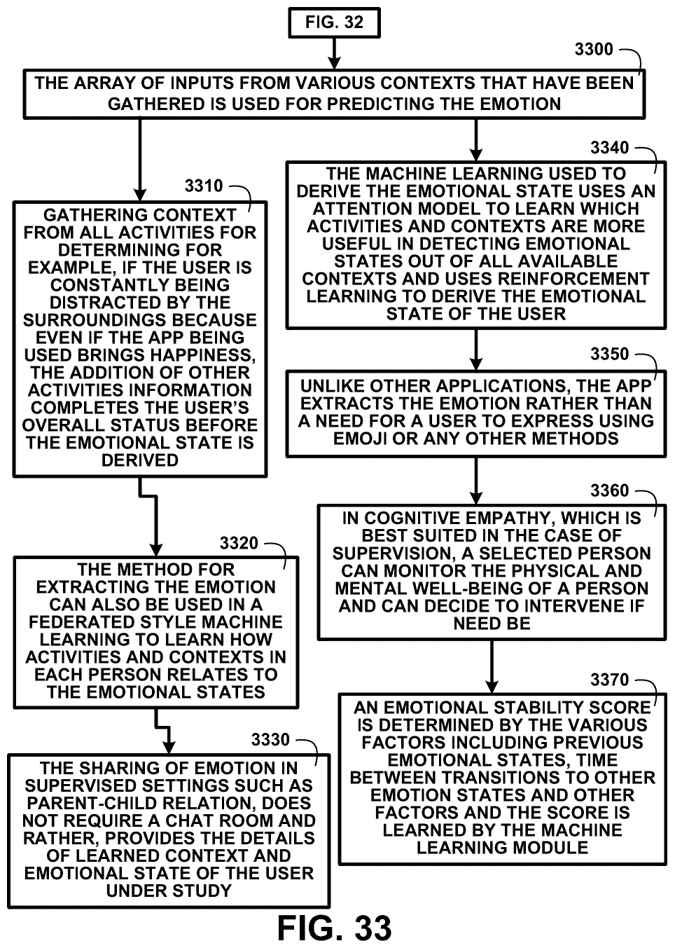

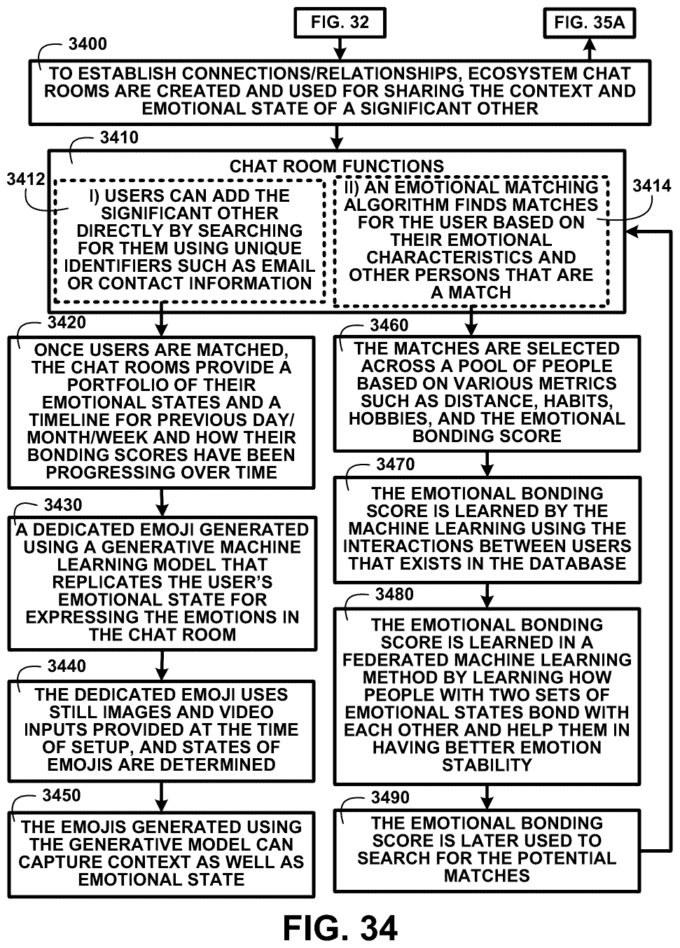

FIG. 32 shows a block diagram of an overview of framework for emotional empathy of one embodiment. FIG. 32 shows the framework for emotional empathy broadly consists of three modules 3200. The first module is capturing the required data for prediction 3210. The second module is a prediction of emotion 3212 with a description that is shown in FIG. 33. The third module is a prediction of emotional bonding and suggestions for improving relation 3214 with a description that is shown in FIG. 34. The first module shows where machine learning is used for capturing the data for context derivation for the required data for prediction 3220. While using an app, the data to be captured is learned by machine learning rather than using a predetermined set of features 3230. Instead of relying on time as a feature, machine learning learns which context data to capture which can later be used for emotion detection 3240. Machine learning output can be thought of as an intermediate output of deep neural networks which will then be utilized by subsequent steps of the network cloud platform 3250.

The context is derived from different activities, for example, when a user is using an app, engaging in social environments, exercising, or other daily user activities 3260. In case of activity detection outside of the handheld devices, the activities are detected by motion sensors, wearable, and a plurality of sensors including imaging, microphones and video feed using handheld devices 3270. A predetermined feature for example, time when directly tracked and used for emotion derivation can result in the inaccurate estimation, where a few apps in the starting might be enjoyable but the app gets boring over time or vice versa 3280 of one embodiment.

### Machine Learning Used to Derive the Emotional State:

FIG. 33 shows a block diagram of an overview of machine learning used to derive the emotional state of one embodiment. FIG. 33 shows a continuation from FIG. 32. The array of inputs from various contexts that have been gathered is used for predicting the emotion 3300. Gathering context from all activities for determining for example, if the user is constantly being distracted by the surroundings because even if the app being used brings happiness, the addition of other activities information completes the user's overall status before the emotional state is derived 3310. The method for extracting the emotion can also be used in a federated style machine learning to learn how activities and contexts in each person relates to the emotional states 3320.

The sharing of emotion in supervised settings such as parent-child relations does not require a chat room and rather, provides the details of learned context and emotional state of the user under study 3330. The machine learning used to derive the emotional state uses an attention model to learn which activities and contexts are more useful in detecting emotional states out of all available contexts and uses reinforcement learning to derive the emotional state of the user 3340. Unlike other applications, the app extracts the emotion rather than a need for a user to express using emoji or any other methods 3350. In cognitive empathy, which is best suited in the case of supervision, a selected person can monitor the physical and mental well-being of a person and can decide to intervene, if need be 3360.

An emotional stability score is determined by the various factors including previous emotional states, time between transitions to other emotion states and other factors and the score is learned by the machine learning module 3370 of one embodiment. Transformer based emotion detection: A transformer is a deep learning model that adopts the mechanism of self-attention, differentially weighting the significance of each part of the input data. The emotional data is a sequence of input from different sensors. The context can be extracted using the special architecture of the transformer for accurate emotion state modelling.

### Ecosystem Chat Rooms:

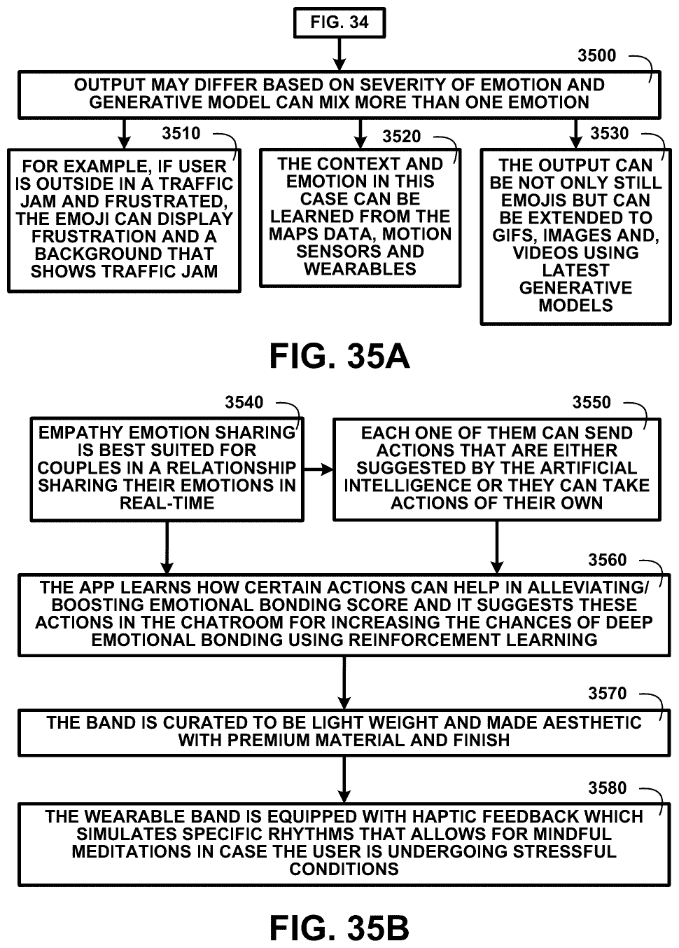

FIG. 34 shows a block diagram of an overview of ecosystem chat rooms of one embodiment. FIG. 34 shows a continuation from FIG. 32 to establish connections/relationships; ecosystem chat rooms are created and used for sharing the context and emotional state of a significant other 3400. Additional descriptions are shown in FIG. 35A. To establish connections/relationships, ecosystem chat rooms are created and used for sharing the context and emotional state of a significant other 3400, while descriptions are detailed in chat room functions 3410. One group of chat room functions 3410 are i) users can add the significant other directly by searching for them using unique identifiers such as email or contact information 3412. Once users are matched, the chat rooms provide a portfolio of their emotional states and a timeline for the previous day/month/week, and how their bonding scores have been progressing over time 3420. A dedicated emoji is generated using a generative machine learning model that replicates the user's emotional state for expressing the emotions in the chat room 3430. The dedicated emoji uses still images and video inputs provided at the time of setup, and states of emojis are determined 3440. The emojis generated using the generative model can capture context as well as emotional state 3450.

Another group of chat room functions 3410 include ii) an emotional matching algorithm finds matches for the user based on their emotional characteristics and other persons that are a match 3414. The matches are selected across a pool of people based on various metrics such as distance, habits, hobbies, and the emotional bonding score 3460. The emotional bonding score is learned by the machine learning using the interactions between users that exists in the database 3470. The emotional bonding score is learned in a federated machine learning method by learning how people with two sets of emotional states bond with each other and help them in having better emotion stability 3480. The emotional bonding score is later used to search for the potential matches 3490 of one embodiment.

### Output May Differ:

FIG. 35A shows a block diagram of an overview of output may differ of one embodiment. FIG. 35A shows a continuation from FIG. 34. Output may differ based on severity of emotion and the generative model can mix more than one emotion 3500. For example, if a user is outside in a traffic jam and frustrated, the emoji can display frustration and a background that shows traffic jam 3510. The context and emotion, in this case, can be learned from the maps data, motion sensors, and wearables 3520. The output can be not only still emojis but can be extended to gifs, images, and videos using the latest generative models 3530 of one embodiment.

### Empathy Emotion Sharing:

FIG. 35B shows a block diagram of an overview of empathy emotion sharing of one embodiment. FIG. 35B shows empathy emotion sharing is best suited for couples in a relationship sharing their emotions in real-time 3540. Each one of them can send actions that are either suggested by the artificial intelligence or they can take actions of their own 3550. The app learns how certain actions can help in alleviating/boosting emotional bonding score and it suggests these actions in the chatroom for increasing the chances of deep emotional bonding using reinforcement learning 3560. The band is curated to be light weight and made aesthetic with premium material and finish 3570. The wearable band is equipped with haptic feedback which simulates specific rhythms that allows for mindful meditations in case the user is undergoing stressful conditions 3580 of one embodiment.

### Children Screen Time Using Gaming Consoles:

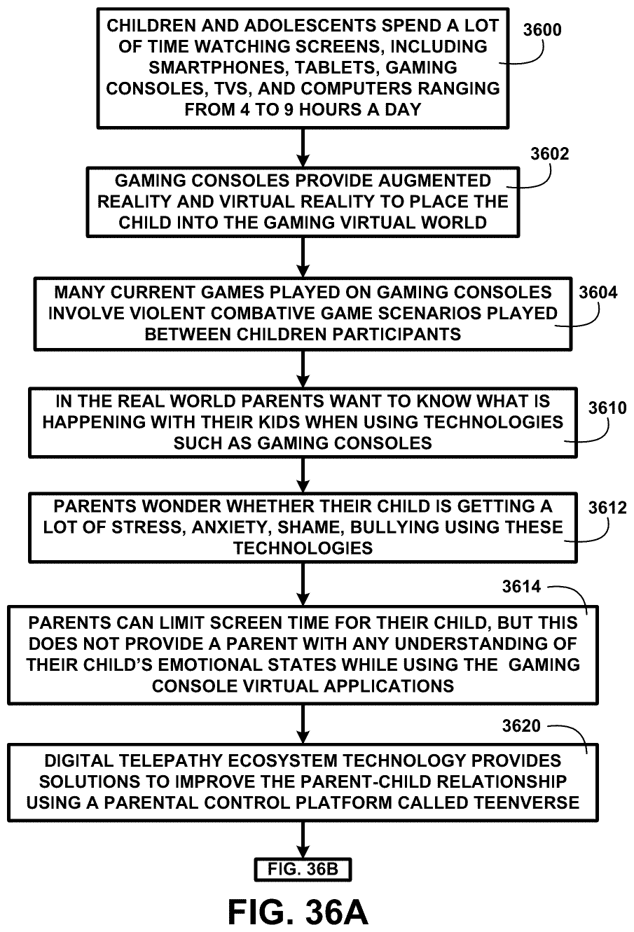

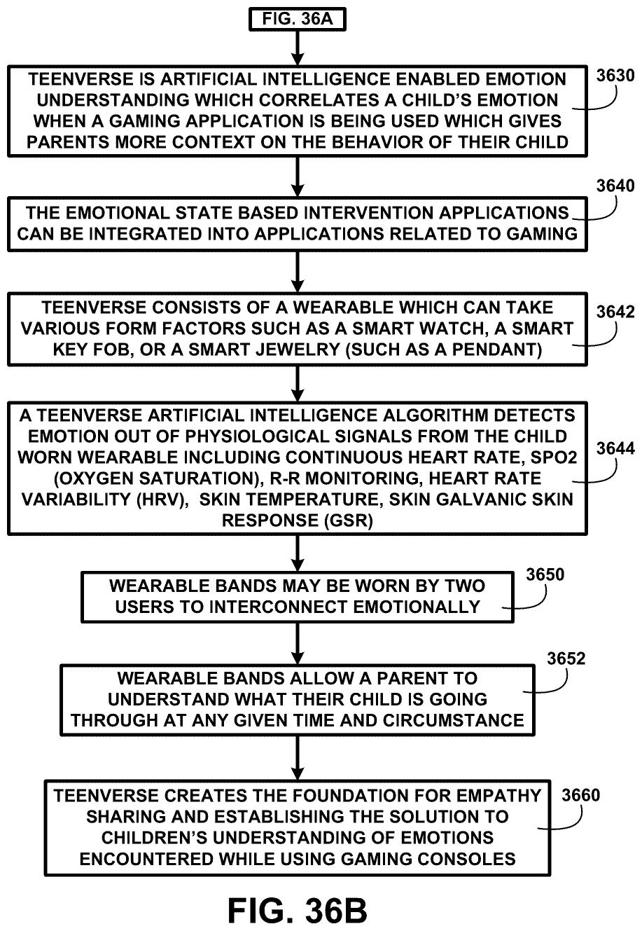

FIG. 36A shows a block diagram of an overview of children screen time using gaming consoles of one embodiment. FIG. 36A shows children and adolescents spend a lot of time watching screens, including smartphones, tablets, gaming consoles, TVs, and computers ranging from 4 to 9 hours a day 3600. Gaming consoles provide augmented reality and virtual reality to place the child into the gaming virtual world 3602. Many current games played on gaming consoles involve violent combative game scenarios played between children participants 3604. In the real world, parents want to know what is happening with their kids when using technologies such as gaming consoles 3610. Parents wonder whether their child is getting a lot of stress, anxiety, shame, and bullying using these technologies 3612. Parents can limit screen time for their child, but this does not provide a parent with any understanding of their child's emotional states while using the gaming console virtual applications 3614. Digital telepathy ecosystem technology provides solutions to improve the parent-child relationship using a parental control platform called teenverse 3620. Descriptions continue in FIG. 36B of one embodiment.

### Teenverse Artificial Intelligence Enabled Emotion Understanding:

FIG. 36B shows a block diagram of an overview of teenverse artificial intelligence enabled emotion understanding of one embodiment. FIG. 36B shows a continuation from FIG. 36A. Teenverse is artificial intelligence enabled emotion understanding which correlates a child's emotion when a gaming application is being used which gives parents more context on the behavior of their child 3630. The emotional state-based intervention applications can be integrated into applications related to gaming 3640. Teenverse consists of a wearable which can take various form factors such as a smart watch, a smart key fob, or a smart jewelry (such as a pendant) 3642. A teenverse artificial intelligence algorithm detects emotion out of physiological signals from the child worn wearable including continuous heart rate, SPO2 (oxygen saturation), R-R monitoring, heart rate variability (HRV), skin temperature, skin galvanic skin response (GSR) 3644. Wearable bands may be worn by two users to interconnect emotionally 3650. Wearable bands allow a parent to understand what their child is going through at any given time and circumstance 3652. Teenverse creates the foundation for empathy sharing and establishing the solution to children's understanding of emotions encountered while using gaming consoles 3660.

The foregoing has described the principles, embodiments, and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

1-30. (canceled)

31. A method, comprising:
displaying advertisements from an advertiser stored on a remote server to a user's digital device;
determining with a wearable device having a plurality of sensors emotional indications of the user in response to the displayed advertisements;
sending the emotional indications from the digital device to a remote server;
comparing the determined user's emotional indications on the server with stored known user's emotional indications associated with particular emotional states to find matching patterns to determine a probable emotional state;
determining indications of the probable emotional state of the user based on the emotional indications;
providing the user with the emotional indications and a probable emotional state based on the emotional indications;
sharing the emotional indications of the probable emotional state with persons selected by the user; and
transferring platform blockchain cryptocurrency rewards from advertisers to the user after the user receives advertisements. related to the probable emotional state.

32. The method of claim 1, further comprising providing advertiser predetermined blockchain cryptocurrency rewards to at least one of the user, a guardian, parents of the user, or a social media contact.

33. The method of claim 1, wherein the user's emotional state includes determining indications of the user's emotional state including at least one of stress, anxiety, sleep disorder, depression, lack of gratitude, and loneliness when viewing the displayed advertisements.

34. The method of claim 1, further comprising providing the user with intervention applications for the user and others to help users overcome troublesome emotions with application integrated interventions including at least one of music, narration, games, and puzzles displayed in at least one advertisement.

35. The method of claim 1, further comprising providing the user with graphical emojis displayed in at least one advertisement associated with emotional states.
